(19) United States
(12) Patent Application Publication
(10) Pub. No.: US 2019/0125701 A1
(43) Pub. Date: May 2, 2019

(54) Compositions and Methods for Treating Viral Infection in Mammals

(71) Applicant: YALE UNIVERSITY, New Haven, CT (US)

(72) Inventors: Anthony van den Pol, Branford, CT (US); Sara Ornaghi, New Haven, CT (US)

(21) Appl. No.: 16/098,535

(22) PCT Filed: May 4, 2017
(86) PCT No.: PCT/US2017/030966

§ 371 Date: Nov. 2, 2018

Related U.S. Application Data
(60) Provisional application No. 62/332,821, filed on May 6, 2016.

(51) Int. Cl. A61K 31/16; A61P 31/22; A61K 35/763

(52) U.S. Cl. CPC A61K 31/16; A61K 31/522; A61K 35/763; A61P 31/22

(57) ABSTRACT

The present invention provides compositions and methods useful for treating and/or preventing Herpesviridae viral infections in a mammal. In certain embodiments, the virus comprises human cytomegalovirus.

### CROSS-REFERENCE TO RELATED APPLICATIONS

The present application claims priority under 35 U.S.C. § 119(e) to U.S. Provisional Application No. 62/332,821, filed May 6, 2016, which application is incorporated herein by reference in its entirety.

### STATEMENT REGARDING FEDERALLY SPONSORED RESEARCH OR DEVELOPMENT

This invention was made with government support under CA161048, CA175577, CA188359 and NS079274 awarded by National Institutes of Health. The government has certain rights in the invention.

### BACKGROUND OF THE INVENTION

A virus is a small infectious agent that replicates only inside the living cells of other organisms. Viruses can infect all types of life forms, from animals and plants to microorganisms. When not infecting a cell or inside an infected cell, viruses exist as viral particles, also known as virions, comprising two or three parts: (i) the genetic material made from either DNA or RNA; (ii) a protein coat, called the capsid, which surrounds and protects the genetic material; and in some cases (iii) an envelope of lipids that surrounds the protein coat when they are outside a cell. The shapes of these virus particles range from simple helical and icosahedral forms for some virus species to more complex structures for others. The average virion is about one one-hundredth the size of the average bacterium.

Viruses exhibit much variation in terms of genetic material within virus particles, and the method by which the material is replicated. Viruses can be, for example DNA viruses (which genome replication takes place in the cell's nucleus), RNA viruses (which replication usually takes place in the cytoplasm; these viruses may be comprise single-stranded (ss) or double-stranded (ds) genetic material); and reverse transcribing viruses (which comprise ssRNA or dsDNA in their particles).

Cytomegalovirus is a genus of viruses in the order Herpesvirales, and in the family Herpesviridae. There are currently eight species in this genus including the type species human herpesvirus 5 (HHV-5), also known as human cytomegalovirus (hCMV).

hCMV is the most common and potentially life-threatening infectious complication in immunocompromised individuals, including AIDS patients and transplant recipients.

CMV infection, which is newly diagnosed in approximately 30,000 U.S. children every year, is particularly damaging to the developing brain (in fetuses and young children) due to the reduced efficacy of the immature innate and systemic immune response to CMV in the immature CNS. CMV is the leading viral cause of congenital birth defects, causing severe problems including myocarditis, pneumonitis, ocular disease, and encephalitis in neonatal mammals and immunocompromised individuals, and congenital abnormalities including microcephaly, cortical thinning, and cerebellar hypoplasia in infected fetuses. This makes CMV the most common severely disabling perinatal infectious agent. CMV infection in the brain is linked to blindness, deafness, lowered IQ and other cognitive and sensory deficit. Further, there appears to be a link between perinatal CMV infection and autism spectrum disorder (ASD) in children and adolescents. Unfortunately, toxicity, modest efficacy, and drug resistance significantly limit the use of current antivirals against CMV. In particular, no treatments are available for congenital CMV infection due to the teratogenicity, acute and long-term toxicity, and carcinogenicity of current anti-CMV drugs. These serious side effects relate to the mechanism of anti-CMV action: inhibition of DNA polymerase. The emergence of drug-resistant CMV strains also poses a challenge, and no effective CMV vaccine is currently available.

There is a need in the art for compounds and/or compositions that can be used to treat and/or prevent viral infections in mammals. Such compounds and/or compositions should be effective against the viral infection with minimal toxicity effects (such as, for example, teratogenesis and liver damage) against the infected mammal. The present invention addresses these needs.

### BRIEF SUMMARY OF THE INVENTION

The invention provides a method of treating and/or preventing an infection by a Herpesviridae family virus in a mammalian subject. The invention further provides a pharmaceutical composition that is useful for treating and/or preventing an infection by a Herpesviridae family virus.

In certain embodiments, the method comprises administering to the subject in need thereof a therapeutically effective amount of at least one compound selected from the group consisting of: valnoctamide (2-ethyl-3-methylpentanamide)

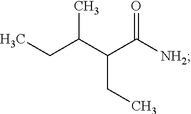

tert-butylethylacetamide (TED; 2-ethyl-3,3-dimethylbutanamide)

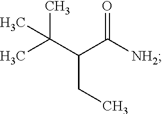

and a molecule of formula (I):

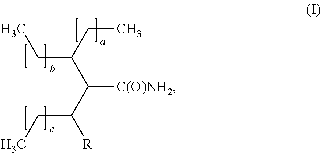

wherein a is 0, 1, 2, 3 or 4; b is 1, 2 or 3; c is 1, 2 or 3; and R is selected from the group consisting of H and C₁-C₄ alkyl; or a solvate, salt, enantiomer, diastereoisomer or any mixtures thereof.

In certain embodiments, a is 0. In other embodiments, R is H or CH₃. In yet other embodiments, R is H, a is 0, b is 1 and c is 1. In yet other embodiments, the compound is the molecule of structure (II), or a solvate, salt, enantiomer, diastereoisomer or any mixtures thereof:

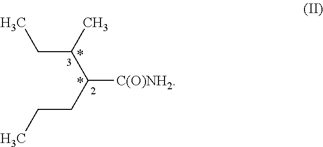

In yet other embodiments, R is CH₃, a is 0, b is 0 and c is 1. In yet other embodiments, the compound is the molecule of structure (III), or a solvate, salt, enantiomer, diastereoisomer or any mixtures thereof:

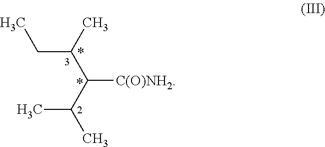

In certain embodiments, (II) is a diastereoisomer selected from the group consisting of (2R,3R), (2R,3S), (2S,3R) and (2S,3S), or any mixtures thereof. In other embodiments, (III) is a diastereoisomer selected from the group consisting of (2R,3R), (2R,3S), (2S,3R) and (2S,3S), or any mixtures thereof. In yet other embodiments, the compound is in an enantiomerically and/or diastereoisomerically pure form. In yet other embodiments, the compound is in an enantiomeric and/or diastereoisomeric mixture. In yet other embodiments, the compound is part of a pharmaceutical composition or formulation further comprising at least a pharmaceutically acceptable carrier.

In certain embodiments, the virus comprises at least one selected from the group consisting of cytomegalovirus (CMV), Epstein-Barr virus (EBV), herpes simplex virus (HSV) 1 and 2, varicella-zoster virus (VZV), and human herpes virus (HHV) 6, 7, and 8. In other embodiments, the virus comprises CMV.

In certain embodiments, the administration of the compound has no significant anticonvulsant effect in the subject. In other embodiments, the administration of the compound has no significant mood stabilizing and/or modifying effect in the subject.

In certain embodiments, the subject is further administered one or more additional agents useful for treating and/or preventing the viral infection. In other embodiments, the one or more additional agents comprise at least one selected from the group consisting of Ganciclovir, Valganciclovir, Foscarnet, Cidofovir, Fomivirsen, Aciclovir, and Valaciclovir. In yet other embodiments, the compound and the one or more additional agents are co-administered to the subject. In yet other embodiments, the compound and the one or more additional agents are coformulated. In yet other embodiments, the compound is administered to the subject by at least one route selected from the group consisting of oral, nasal, inhalational, topical, buccal, rectal, pleural, peritoneal, intra-peritoneal, vaginal, intramuscular, subcutaneous, transdermal, epidural, intratracheal, otic, intraocular, intrathecal, intra-amniotic, intra-umbilical cord, and intravenous routes. In yet other embodiments, the therapeutically effective amount of the compound ranges from about 0.001 mg/day to about 10,000 mg/day.

In certain embodiments, the subject is human. In other embodiments, the subject is pregnant. In yet other embodiments, the subject is neonatal. In yet other embodiments, the subject is immunocompromised. In yet other embodiments, the subject is an unborn fetus. In yet other embodiments, the female carrying the fetus is immunocompromised.

In certain embodiments, the pharmaceutical composition comprises at least one compound selected from the group consisting of VCD, TED, and a molecule of formula (I):

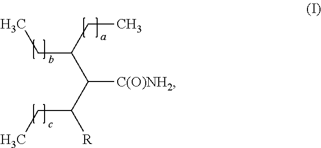

wherein a is 0, 1, 2, 3 or 4; b is 1, 2 or 3; c is 1, 2 or 3; and R is selected from the group consisting of H and C₁-C₄ alkyl, or a solvate, salt, enantiomer, diastereoisomer or any mixtures thereof; and one or more additional agents useful for treating and/or preventing a viral infection.

In certain embodiments, the viral infection comprises a virus from the Herpesviridae family. In other embodiments, the virus comprises CMV. In yet other embodiments, the at least one compound is selected from the group consisting of valnoctamide, 2-ethyl-3,3-dimethylbutanamide (TED), 2-isopropyl-3-methylpentanamide (SID) and 3-methyl-2-propylpentanamide (SPD), or a solvate, salt, enantiomer, diastereoisomer or any mixtures thereof.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIGS. 1A-1E illustrate the finding that valproate and valpromide exert opposing effects on mCMV. FIG. 1A: Representative microscopic fields show CMV GFP reporter fluorescence (top) and phase contrast (bottom) of NIH/3T3 cells pre-treated (24 hrs) with VPA (1 mM) or vehicle prior to inoculation with mCMV using multiplicity of infection (MOI) of 0.4. Photos captured 48 hpi; scale bar 50 μm. FIG. 1B: Bar graph shows VPA dose-dependent increase in mCMV infection, other conditions same as A. FIG. 1C: VPD (1 mM) with other conditions same as FIG. 1A. FIG. 1D: Bar graph shows VPD dose-dependent decrease in mCMV infection, other conditions same as FIG. 1A. FIG. 1E: NIH/3T3 cells exposed to VPD or vehicle for 4, 24, and 72 hrs prior to viral inoculation, other conditions same as FIG. 1A. Time-dependent pre-incubation drug-induced decrease in mCMV infection is shown. FIG. 1B, FIG. 1D, FIG. 1E: Mean±SEM of 8 cultures. ***p<0.001, **p<0.01, as compared to control, ANOVA with Bonferroni's post-hoc test. In FIG. 1E, p<0.01, 72 hrs-versus 24 hrs-pretreatment; p<0.001, 72 hrs—versus 4 hrs—pretreatment.

FIGS. 2A-2E illustrate the finding that valnoctamide inhibits both mCMV and hCMV. FIG. 2A: Microscopic fields show GFP fluorescence (top) and phase contrast (bottom) of NIH/3T3 cells pre-treated (24 hrs) with VCD (1 mM) or vehicle prior to inoculation with mCMV (MOI 0.4). Photos captured 48 hpi; scale 50 μm. FIG. 2B-2C: Dose—(FIG. 2B) and time-dependent (FIG. 2C) VCD-mediated decrease in mCMV infection (MOI 0.4). FIG. 2D: VCD dose-dependent decrease using high MOI (4.0) mCMV. Infected cells counted 24 hpi, other conditions same as A. FIG. 2E: VCD dose-dependent decrease using normal human dermal fibroblasts inoculated with hCMV (MOI 0.1). Infected cells counted 72 hpi, other conditions same as A. FIGS. 2B-2E: Mean±SEM of 8 cultures. ***p<0.001, **p<0.01, *p<0.05, one-way ANOVA, Bonferroni's post-hoc test. In FIG. 2E, p<0.01, 72 hrs—versus 24 hrs—pretreatment; p<0.001, 72 hrs—versus 4 hrs—pretreatment.

FIGS. 3A-3N illustrate the finding that valpromide and valnoctamide inhibit mCMV in vivo and significantly improve post-natal development of infected newborns. FIG. 3A: Timeline showing mCMV infection of neonates and compound administration. DOB, date of birth; PND, postnatal day; i.p., intraperitoneal; s.c., subcutaneous. FIG. 3B: Survival at PND 49 assessed by Log-rank (Mantel-Cox) test; N=11-13 mice/group. FIGS. 3C-3J: Improvement in postnatal somatic development. Photo shows improved growth of VPD- and VCD-treated pups compared to vehicle (VEH, mCMV) (FIG. 3C). Graphs show mean±SEM; error bars shown for VEH group (FIG. 3D-3F) or not shown (FIG. 3G-3J) for clarity. N=6-9 mice/group; mixed-model ANOVA (Newman Keuls test); VPD and VCD versus CTR (control/uninfected newborns) (FIG. 3D), and for VPD and VCD versus VEH (FIG. 3D-3J). FIG. 3K: Photos highlight delayed development of fur in an infected/untreated pup (left) compared to an infected newborn treated with VCD (right). Marked difference in growth is also evident. FIG. 3L: Photo shows the differential status of eye opening. FIGS. 3M-3N: Infected newborns treated with VPD, VCD, or VEH from PND 1 to 10, were euthanized at PND 12, and tissue samples from liver and lungs were collected for viral titration by plaque assay. Viral titers of individual mice (symbols) and median values (horizontal bars) are shown; dotted lines represent limit of detection; ns, not significant, *p<0.05, **p<0.01, ***p<0.001, one-way ANOVA, Bonferroni's post-hoc test.

FIGS. 4A-4I illustrate the finding that valpromide and valnoctamide inhibit CMV binding to cells. FIGS. 4A-4B: NIH/3T3 cells infected with mCMV-GFP (MOI 0.1) (t=0) were exposed to VPD, VCD, or vehicle (100 μM) simultaneously (0-48 hpi), or 2 hours (2-48 hpi) or 12 hours (12-48 hpi) after virus inoculation. Photos captured of GFP-positive cells counted 48 hpi. In FIG. 4A, scale 100 μm. FIG. 4C: Murine fibroblasts infected with mCMV (MOI 0.1) received VPD, VCD, or vehicle (100 μM) simultaneously to viral challenge. After 2 hrs-incubation, cultures were rinsed 3× with PBS, and replaced with fresh drug-free media. Results collected at 48 hpi. FIG. 4D: A drug (100 μM)/undiluted mCMV mixture was incubated for 2 hours at 37° C. or 4° C. Before cell inoculation, the solution was diluted to 10 nM (ineffective drug concentration). FIGS. 4E-4F: Pictogram details experiments addressing questions related to CMV binding and fusion (FIG. 4E). Infectivity assessed at 48 hpi; HS, heparan sulfate (FIG. 4F). FIG. 4G: After 24 hrs-VPD, VCD, or vehicle pre-treatment, cultures were rinsed twice and given drug-free media prior to viral inoculation (MOI 0.1). Bars: mean±SEM of 8 cultures (FIGS. 4B-4D and 4G) and 12 cultures (FIG. 4F). ***p<0.001, **p<0.01, one-way ANOVA with Bonferroni's post-hoc test. FIGS. 4H-4I: Plaque assay of mCMV-infected NIH/3T3 cells (MOI 1) with VPD, VCD, or vehicle. Viral plaque size measured 5 dpi. Representative plaques in 100 μM VCD (top) or vehicle (bottom); scale 300 μm (FIG. 4H). Mean diameter of 60 random plaques; p<0.05 in 0.1 μM, p<0.01 in 1 μM, p<0.001 in 100 μM and 1 mM, versus vehicle. SEM, bar on upper right side (FIG. 4I).

FIGS. 5A-5F illustrate the finding that valpromide inhibits mCMV infection in different cell types and decreases viral-mediated cell death. FIG. 5A: Images of representative microscopic fields under GFP fluorescence (top) and phase contrast (bottom) of NIH/3T3 cells pre-treated (24 hrs) with VPD or vehicle (10 mM) prior to inoculation with high titer mCMV-GFP (MOI 4). Photos captured at 60 hpi; scale bar 100 μm. FIGS. 5B-5D; Bar graph shows VPD dose-dependent decrease in mCMV infection in NIH/3T3 (FIG. 5B), immortalized Neuro-2a (FIG. 5C) and primary mouse glia (FIG. 5D) cells, other conditions same as FIG. 5A. Infectivity assessed 24 hpi by counting GFP-positive cells. FIGS. 5E-5F: The protective effect of VPD on viral-mediated cell cytotoxicity was assessed by the red fluorescent ethidium homodimer (EthD-1) assay. Images show red fluorescent photomicrographs of NIH/3T3 cells pre-treated with VPD or vehicle at 10 mM for 24 hrs prior to viral inoculation (MOI of 0.4). 72 hpi, EthD-1 was added to cells. After 20 minutes, photos were collected (FIG. 5E) and red fluorescent-labeled cells were counted (FIG. 5F). In FIG. 5E, scale bar 50 μm. Data presented as mean±SEM of 8 cultures; ***p<0.001, **p<0.01, and *p<0.05 as compared to control using ANOVA with Bonferroni's post-hoc test.

FIGS. 6A-6C illustrate the finding that valpromide inhibits human CMV. FIG. 6A: Normal human dermal fibroblasts were treated with VPD or vehicle at the indicated concentrations for 24 hrs prior to hCMV-GFP inoculation (MOI 0.1). Results read at 72 hpi. FIG. 6B: Human glioblastoma cells were exposed to VPD or vehicle at 10 mM, 3 mM, 1 mM, 300 μM, 100 μM, and 30 μM for 48 hrs prior to viral inoculation (MOI 4). GFP-positive cells were counted at 48 hpi. Data presented as mean±SEM of 8 cultures (FIG. 6A) and 6 cultures (FIG. 6B). ***p<0.001, *p<0.05, one-way ANOVA (Bonferroni's post-hoc test). FIG. 6C: Immunostaining for hCMV gB was done to exclude a potential inhibitory effect of VPD on GFP expression. Human dermal fibroblasts were exposed to VPD or vehicle (1 mM) for 24 hrs prior to viral challenge (MOI 1). Representative fields with hCMV gB immunoreactivity in red and cell nuclei in blue (DAPI) show that VPD reduced the relative number of infected cells, corroborating VPD-mediated inhibition of hCMV (56%±7% compared to control; mean±SEM, n=5). Scale bar 50 μm.

FIG. 7 illustrates the finding that valpromide had no effect on Vesicular Stomatitis Virus infection. Images of representative microscopic fields under GFP fluorescence (top) and phase contrast (bottom) of Vero cells pre-treated with VPD or vehicle at 1 mM for 24 hrs and infected with VSV-GFP (MOI 0.001). No drug-mediated inhibitory effect was identified (101%±5%, compared to control; mean±SEM of 6 cultures). Photos captured at 24 hpi; scale bar 50 μm.

FIG. 8 illustrates the finding that short exposure to valpromide and valnoctamide significantly decreases virus production. Murine fibroblasts infected with mCMV-GFP (MOI 0.1) received VPD, VCD, or vehicle (100 μM) simultaneously to viral challenge. After 2 hrs incubation, cultures were rinsed twice with PBS, and replaced with fresh drug-free media. 72 hpi cell culture supernatants were collected and plaque-titrated on NIH/3T3 cells. Data presented as mean±SEM of 6 cultures; ***p<0.001, one-way ANOVA with Bonferroni's post-hoc test.

FIG. 9 illustrates the finding that valpromide and valnoctamide appear safe in uninfected newborns. Uninfected pups received 20 μL of saline (CTR), vehicle (VEH), VPD, or VCD (1.4 mg/mL), once a day, subcutaneously, from PND 1 to PND 20, when the body weight was assessed. Bars represent mean±SEM, one-way ANOVA; N=8 mice/experimental group.

FIG. 10 comprises a graph illustrating that CMV plaque size is reduced by VPD, VCD, SPD, and SID. CMV infection plaque sizes were measured in the presence of varying concentrations of four anti-CMV drugs, as well as VPA, which does not attenuate CMV. All four drugs were effective. SPD showed a slightly greater efficacy in vitro.

FIGS. 11A-11C illustrate the finding that sec-butylpropylacetamide inhibits mCMV in vivo and significantly improves survival and post-natal development of CMV-infected newborns. FIG. 11A: Survival at PND 30 assessed by Log-rank (Mantel-Cox) test; N=9-13 mice/group. FIGS. 11B-11C: Improvement in postnatal somatic development. FIG. 11B illustrates improved growth of SPD-treated pup (left) compared to vehicle (VEH, mCMV, right). FIG. 11C: Graph shows mean±SEM; N=9 mice/group; *p<0.05, **p<0.01, ***p<0.001, mixed-model ANOVA (Newman Keuls test); significance for SPD versus CTR (control/uninfected newborns) shown at the top of the graph, and for SPD versus VEH shown at the bottom of the graph.

FIGS. 12A-12E illustrate kinetics of mCMV replication after intraperitoneal inoculation on day of birth. Newborn mice were infected on the day of birth (DOB, day 0) with 750 PFU of mCMV. Viral load in whole blood, liver, spleen, and brain was evaluated by quantitative PCR (qPCR) at the indicated time-points and expressed as log₁₀ genome copies per gram/mL of harvested tissue/blood. In FIG. 12A, each symbol represents an individual mouse and horizontal bars show mean values of the groups; in FIGS. 12B-12E, data are presented as mean±SEM with 7-10 mice/time-point. Viral titers below the limit of detection (LoD, dotted line) were plotted as 2 log₁₀ genome copies. In FIG. 12A, **p<0.01, ***<0.001, ****<0.0001; one-way ANOVA with Bonferroni's post-hoc test; dpi, days post-infection.

FIG. 13 illustrates scattered widespread distribution of mCMV-GFP in brains after infection of newborn mice. Detection of virus-infected cells by means of mCMV GFP reporter expression in representative coronal sections of postnatal day 8 (P8) and P12 mouse brains. Single infected cells or small foci of infection can be identified in the retrosplenial cortex (RS ctx), primary and secondary somatosensory cortex (S1/S2), ectorhinal cortex (Ect), perirhinal cortex (Prh), piriform cortex (Pir), hippocampus (hippo) and dentate gyrus (DG), lateral ventricle (LV), external and internal capsule of the corpus callosum (ec and ic, respectively), lateral hypothalamic area (LH), and thalamic nuclei (Th Nu) of a P12 mouse brain. D3V, dorsal third ventricle (Panel A). Panels B-D are magnification of the boxed areas in Panel A. Infection of the lateral ventricle and diffusion to the adjacent brain parenchyma in a P8 brain (Panel E). In Panel F, magnification of the boxed area in panel E. cc, corpus callosum. Photomicrograph of a P12 brain showing infection in the motor (M1) and piriform cortex, and in the striatum (CPu, caudate putamen) (Panel G). Large foci of mCMV-infected cells in the pons and the medulla of a P8 animal (Panels H-I). Scale bar 50 μm (Panel H), 100 μm (Panels A, D, E, G, I), 200 μm (Panels C and F), and 400 μm (Panel B).

FIG. 14 illustrates CMV infection of neuronal cells in the cerebellum, hippocampus, and cortex of the developing brain. Photomicrographs show GFP labeling of different cerebellar cell types, including neurons in the internal granular layer (Panel A) and Purkinje cells (Panel B), as assessed by NeuN and Calbindin D-28K staining at 8 dpi. Photographs display infection of different areas of the hippocampus (Panel C), a magnification of the viral involvement of pyramidal cells in CA1 field (boxed area) (Panel D), and infected neurons in the dentate gyms (DG) (Panel E). Robust GFP expression in a pyramidal neuron of the motor cortex (Panel F); note the beaded aspect of the basilar dendrites, sign of neuronal pathology. Photomicrograph of neuronal infection in the visual cortex (Panel G). Scale bar 100 μm (Panels A-E, G), 50 μm (Panel F).

FIGS. 15A-15B illustrate the finding that valnoctamide suppresses mCMV load in the brain of mice infected intraperitoneally on the day of birth. Newborn mice were infected at P0 with 750 PFU of mCMV i.p. and randomized to receive either vehicle (mCMV+VEH) or VCD (mCMV+VCD) subcutaneously from P1 until P21. Viral load was quantified in the cerebrum and the cerebellum separately by qPCR at the specified time-points and expressed as log₁₀ genome copies per gram of harvested tissue. Data are presented as mean±SEM; n=7-10 mice/time-point. Viral titers below the limit of detection (LoD, dotted line) were plotted as 2 log₁₀ genome copies. Ns, not significant, *p<0.05, **<0.01, ***<0.001, ****<0.0001; two-way ANOVA with postnatal day as repeated measures.

FIG. 16 illustrates the finding that Subcutaneously injected valnoctamide enters the brain and suppresses mCMV replication within the brain. Quantification of mCMV load in the brain of mice intracranially infected with 2×10⁴ PFU of mCMV on day 3 after birth. The amount of virus in the cerebrum (left) and the cerebellum (right) was calculated by qPCR in P9 mice receiving either vehicle (VEH) or VCD subcutaneously from P3 through P8 and expressed as genome copies per gram of harvested tissue. Mean±SEM; n=8 mice/time-point. **<0.01, ***<0.001, Mann-Whitney U-test.

FIGS. 17A-17H illustrate the finding that delayed acquisition of neurological milestones induced by mCMV infection is completely rescued by valnoctamide therapy. Graphs (x-axis is postnatal day) show neurodevelopmental delays in mCMV-infected pups (solid grey triangles) as assessed by the righting reflex (FIG. 17A), the cliff aversion (FIG. 17B), the forelimb grasping and placing reflex (FIGS. 17C-17D), the negative geotaxis (FIG. 17E), the level screen test (FIG. 17F), the screen climbing test (FIG. 17G), and the vibrissa placing reflex (FIG. 17H). mCMV-infected VCD-treated animals (solid green triangles) showed neurological responses similar to uninfected controls receiving either vehicle (VEH, empty grey circles) or VCD (empty green circles). Mean±SEM, n=20-24 mice (9-12 males)/experimental group; ns, not significant, *p<0.05, **<0.01, ***<0.001, ****<0.0001; two-way ANOVA with postnatal day as repeated measures. Significance shown next to infected, untreated mice (mCMV+VEH) line for comparison with uninfected controls (CTR+VEH and CTR+VCD), and next to control lines for comparison with VCD-treated infected pups (mCMV+VCD).

FIGS. 18A-18E illustrate the finding that impaired cerebellar-mediated motor functions in mCMV-infected mice are ameliorated by valnoctamide treatment. Photographs display stereotypical clasping response with hindlimbs retracted to the abdomen in a mCMV-infected mouse (middle), and normal response with splayed out hindlimbs in an uninfected control (left) and in an mCMV-infected, VCD-treated animal (right) (FIG. 18A). Scoring of clasping response according to hindlimb position (FIG. 18B). Increased locomotor activity time (T_{LA}) in infected, untreated mice in the vertical pole test, as compared to VCD-treated infected animals and uninfected controls (FIG. 18C). Investigation of fine motor coordination and balance by challenging beam traversal test. Infected mice need more time to traverse the beam (FIG. 18D) and slip more (FIG. 18E) than the control mice. Both aspects are improved by VCD administration. Mean±SEM; n=10-13 mice/group. *p<0.05, **<0.01, ***<0.001, ****<0.0001; Kruskal-Wallis with Dunn's post-hoc test in FIGS. 18B-18D, two-way ANOVA with Repeated Measures and Bonferroni's post-hoc comparison in FIG. 18E.

FIGS. 19A-19D illustrate the finding that CMV infection during early development causes disturbances in social behavior and exploratory activity in adolescent mice. Sociability (FIG. 19A) and preference for social novelty (FIG. 19B) assessment in infected and control mice, with or without VCD treatment, by means of the three-chamber test. CMV-infected mice display regular sociability compared to control mice but lack a preference for a novel mouse over a known mouse. This lack of preference for social novelty is restored by VCD administration. Exploratory activity was assessed by quantification of rearing (FIG. 19C) and nose-poking (FIG. 19D) events in a novel environment. The altered exploratory behavior with decreased number of events identified in mCMV-infected animals is rescued by VCD. Mean±SEM; n=10-13 mice/group for social behavior, n=18-22 mice/group for exploratory activity; ns, not significant, *p <0.05, **<0.01, ***<0.001, ****<0.0001; two-way ANOVA with Repeated Measures and Bonferroni's post-hoc comparison in FIGS. 19A-19B), Kruskal-Wallis with Dunn's post-hoc test in FIGS. 19C-19D.

FIGS. 20A-20B illustrate the finding that valnoctamide reverses deficient brain growth induced by mCMV infection. Photograph shows decreased brain size in an infected, untreated mouse (mCMV, middle), compared to an uninfected control (left). VCD treatment restores normal brain growth (mCMV+VCD, right) (FIG. 20A). Quantification of VCD-mediated benefits on postnatal brain growth by calculation of brain to body weight ratio. Mean±SEM; n=10 mice/group (3 litters); ns, not significant, **p<0.01, ***<0.001; one-way ANOVA with Bonferroni's post-hoc test (FIG. 20B).

FIGS. 21A-21H illustrate the finding that valnoctamide substantially ameliorates cerebellar development in mCMV-infected mice. Photomicrograph of representative fluorescent Nissl-stained cerebellar areas in control (left) and infected mice with (right, mCMV+VCD) or without (middle, mCMV) VCD. Note the delayed foliation in infected, untreated cerebellum, rescued by VCD; scale bar 200 μm (FIG. 21A). Graph depicts cerebellar area, expressed as percentage of total brain area (3 sagittal sections/animal, 5 animals/group, 3 litters) (FIG. 21B). Photomicrograph showing cerebellar Purkinje cells (PCs) and molecular layer (ML) by means of Calbindin D-28K staining. Infected, untreated cerebellum (middle) displays loss of PCs and thinner ML compared to uninfected control (left); VCD improves both parameters (right). Scale bar 200 μm (FIG. 21C). Quantification of PC number (FIG. 21D), and ML (FIG. 21E) and internal granular layer (IGL) thickness (FIG. 21F) along 500 μm of the primary fissure (prf, both sides) (3 sagittal sections/mouse, 5 mice/group, 3 litters). Fluorescent micrograph of heterotopic PCs (arrowheads) identified in an infected untreated cerebellum; scale bar 100 μm (FIG. 21G). Photomicrograph displays pathological persistence of external granular layer (EGL) in mCMV-infected, untreated cerebellum at P30 (middle); no EGL could be identified at the same time-point in uninfected control (left) and infected, VCD-treated cerebellum (right); scale bar 200 μm (FIG. 21H). Mean±SEM; ns, not significant, *p<0.05, **<0.01, ***<0.001; one-way ANOVA with Bonferroni's post-hoc test.

FIGS. 22A-22C illustrate the finding that valnoctamide suppresses hCMV infectivity and replication in human fetal astrocytes by blocking virus attachment to the cell. Human fetal astrocyte cells were pre-treated (1 h) with VCD (100 μM) or vehicle (VEH) prior to inoculation with hCMV using MOI of 0.1. VCD treatment decreased hCMV infectivity and replication as assessed by GFP-positive cell counting (FIG. 22A) and viral yield assay (FIG. 22B) at 48 hpi. Viral inoculated human fetal astrocytes were exposed to VCD or vehicle (100 μM) for 1 h at either 4° C. or 37° C. to assess hCMV attachment to (Bound virus') and internalization into (‘Internalized virus’) the cell. Viral DNA was quantified by qPCR and results expressed as % of control (vehicle-treated cultures considered as 100%) (FIG. 22C). Graphs represent the average of three separate experiments each performed in triplicate, error bars correspond to standard error. ns, not significant, ***p<0.001, ****<0.0001, unpaired Student's t-test in FIGS. 22A and 22C), Mann-Whitney U-test in FIG. 22B; in FIG. 22C significance refers to the comparison between VCD- and vehicle-treated cultures in each assay.

### DETAILED DESCRIPTION OF THE INVENTION

The invention relates in certain aspects to the unexpected discovery that certain compounds can be used to treat and/or prevent a Herpesviridae virus infection in a mammal. In certain embodiments, the virus comprises cytomegalovirus (CMV). In other embodiments, the virus comprises Epstein-Barr virus (EBV). In yet other embodiments, the virus comprises a virus belonging to the Herpesviridae family, such as but not limited to, CMV, EBV, herpes simplex virus (HSV) 1 and 2, varicella-zoster virus (VZV, also called chicken pox), and human herpes virus (HEW) 6, 7, or 8. In yet other embodiments, the virus is not vesicular stomatitis virus. In yet other embodiments, the virus is not Sindbis virus.

The present invention includes methods of treating and/or preventing a Herpesviridae virus infection in a subject. In certain embodiments, the invention includes methods of treating a CMV infection in a subject. In other embodiments, the subject is pregnant. In yet other embodiments, the subject is neonatal. In yet other embodiments, the subject is an unborn fetus. In yet other embodiments, the subject is human.

Valproate (VPA) is a widely used anti-epileptic drug, employed for the treatment of multiple psychiatric and neurological diseases, including bipolar disorder, epilepsy, neuropathic pain, and migraine. Significant side effects of VPA therapy include liver toxicity and teratogenesis. The inhibitory action of VPA on histone deacetylases (HDACs) underlie the detrimental effects exerted by this drug on neural tube defects, skeletal abnormalities, and autism during fetal development. Valpromide (VPD), a more effective and less toxic anti-epileptic homologue of VPA, has been used as a mood-stabilizer in bipolar disorder for over 25 years. In contrast to VPA, VPD lacks HDAC inhibitory activity.

Although VPA and VPD attenuate reactivation from latency of Epstein Barr virus (EBV), VPA enhances infectivity and replication of a large variety of other viruses including HIV, VSV, Kaposi's sarcoma-associated herpes virus, and the beta-herpes viruses HHV-6 and hCMV through a mechanism involving HDAC inhibition. These enhancing effects raise concerns over the use of VPA in AIDS patients for treatment of neurological disorders and in congenitally CMV-infected neonates experiencing seizures.

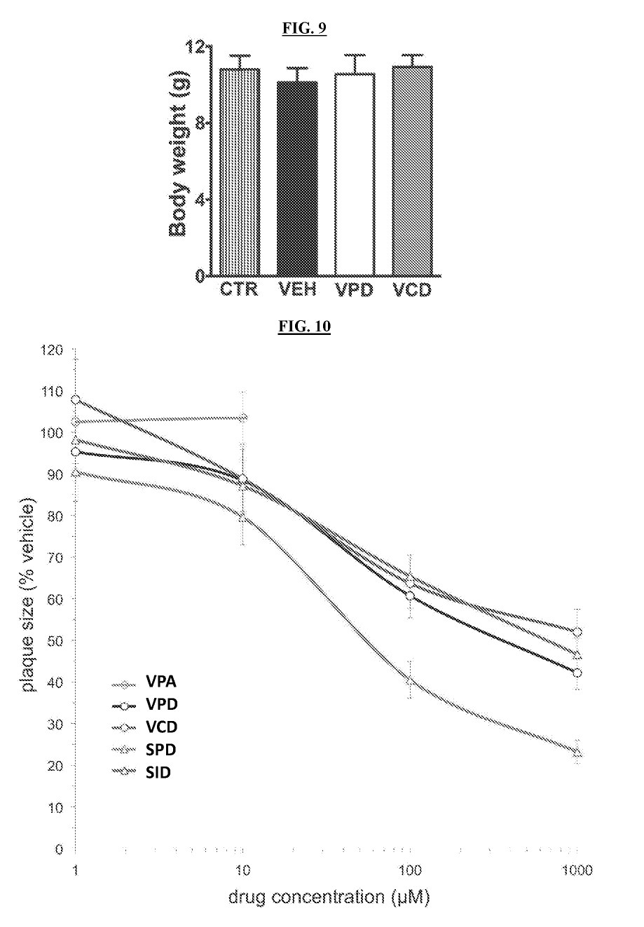

As demonstrated herein, VPD and VCD, which are drugs used for many years to treat neurological disorders, evoke an unexpected, substantial, and specific inhibition of both human and mouse CMV both in vitro and in vivo. In certain non-limiting embodiments, the drugs act by blocking an early phase of CMV infection. The strong anti-CMV activity of these drugs is substantiated by multiple converging lines of evidence including reduction in infected cell number as determined with both GFP reporter expression and immunocytochemistry, reduction in cell death, reduction in virus plaque size, increase in in vivo survival and reduction in CMV-induced health deficits. Anti-CMV activity of these compounds had never been described. CMV inhibition takes place at low drug concentrations below the therapeutic levels used for anti-convulsant and mood stabilizing purposes. Furthermore, drug administration to uninfected newborn mice evoked no adverse response (FIG. 9). In certain embodiments, VCD, safe for use in humans, can be used to reduce severe problems caused by CMV infection during development and in conditions of reduced systemic immunity.

As demonstrated herein, administration of VCD during the first 3 weeks of life restored timely acquisition of neurological milestones in neonatal mice and rescued long-term motor and behavioral outcomes in juvenile mice. CMV-mediated brain defects, including decreased brain size, cerebellar hypoplasia, and neuronal loss, were substantially attenuated by VCD. No adverse side effects on neurodevelopment of uninfected control mice receiving VCD were detected. These data indicate that VCD during critical periods of neurodevelopment can effectively suppress CMV replication in the brain and safely improve both immediate and long-term neurological outcomes. Treatment of CMV-infected human fetal astrocytes with VCD reduced both viral infectivity and replication by blocking viral particle attachment to the cell, a mechanism that differs from available anti-CMV drugs.

As demonstrated herein, low-dose VCD administered outside the brain during early ontogeny effectively suppresses mCMV inside the developing brain of infected mice via at least two different sites of action. VCD reduces peripheral levels of mCMV, thereby decreasing the amount of virus available for entry into the brain. Secondly, VCD acts directly within the brain to block existing brain CMV infection. The dose that blocks CMV in the present studies is lower than the dose used to attenuate seizures in neonatal rodent experiments. The antiviral action of VCD begins shortly after administration and effectively attenuates CMV levels throughout the brain during the critical period of postnatal brain development. This decrease in viral load is accompanied by a concomitant restoration of normal early neurological outcomes in infected neonatal mice treated with VCD. Late-onset neurobehavioral dysfunction, including motor impairment and social and exploratory behavior disturbances, as well as virally induced deficient brain growth and disrupted cerebellar development, are rescued in CMV-infected adolescent mice that received VCD during the neonatal period, suggesting long-lasting beneficial effects. No adverse collateral effects on the neurodevelopment of uninfected control mice treated with VCD was observed.

The newborn mouse brain is substantially less developed than the newborn human brain. Based on the timing of the brain growth spurt, initial neurogenesis, establishment and refinement of connections, myelination, and gliogenesis, the mouse CNS at birth is proposed to parallel the late first/early second-trimester human fetal CNS. This is a critical period for human brain development and for hCMV infection. By infecting mouse pups on the day of birth, this animal model provides an informative means to study the effects of CMV on the developing brain. Infected newborn mice display similar brain pathology and neurological symptoms to that reported in congenitally infected human infants, including microcephaly, cerebellar hypoplasia, neuronal loss, neurodevelopmental delays, motor impairments, and behavioral disturbances. These data support the validity of this in vivo model for investigating CMV infection and novel anti-CMV treatments during early brain development.

Despite being partially effective, currently available CMV antivirals, including ganciclovir and its prodrug valganciclovir, foscarnet, cidofovir, and fomivirsen, display both toxic and teratogenic actions. For this reason, they are not approved or recommended for the treatment of pregnant women or infected fetuses or neonates, thus depriving those who may need it the most, or at best delaying treatment and hindering potential prevention or amelioration of CMV-induced brain defects during early brain development. Because less severely infected human infants are also at risk for late-onset neurological complications including cognitive and motor disabilities, behavioral disturbances, visual deficits, and hearing impairment, development of anti-CMV compounds with safer in vivo profiles that can be used in all infected neonates would be of substantive benefit.

VCD has shown no teratogenic or toxic activity in several studies employing different animal models of early development, and has been safely used for many years to treat neuropsychiatric disorders in adults. Further confirmation of its safety profile has derived from pre-clinical and clinical investigations of drug-mediated anti-convulsant and mood stabilizing actions. VCD is effective at a low μM dose level, a slightly reduced level of efficacy compared with ganciclovir; nonetheless, substantial CMV inhibition in vivo was observed with subcutaneous delivery. Studies on newborn mice identified potent anti-CMV actions of VCD.

The species-specificity of CMV replication prevents testing the activity of novel antivirals on hCMV in animal models. Murine and human CMV share similar genomes and anti-CMV drugs effective against mCMV are likely to also be active against hCMV. The attenuation of hCMV infection of human fetal astrocytes by VCD corroborates the utility of the present mouse in vivo model, and suggests that VCD is also effective against hCMV in the developing and adult human brain. Without wishing to be limited by any theory, VCD appears to act by blocking hCMV attachment to the cell membrane, a mechanism of action different than that of hCMV antivirals currently available for treatment. In certain non-limiting embodiments, VCD is a therapeutic option in immunocompromised adults, for whom the emergence of drug resistant CMV strains has become a substantial challenge. Other closely related molecules, for instance, valpromide, may also attenuate CMV, but because valpromide can be metabolized to valproate which can enhance virus infections, VCD is a better alternative due to the absence of conversion to valproate.

As demonstrated herein, subcutaneous low-dose VCD effectively and safely attenuates mCMV replication in the developing mouse brain and rescues these animals from virally induced brain defects and adverse neurological outcomes. Further, VCD suppresses hCMV replication in human fetal brain cells by blocking viral attachment to the cell surface. Considering that VCD is already clinically available, has proven safe in multiple models of early development, and displays a novel mechanism of anti-CMV action, it can be used therapeutically to treat CMV infection in the developing human brain.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described. As used herein, each of the following terms has the meaning associated with it in this section.

Generally, the nomenclature used herein and the laboratory procedures in cell culture, oncology, molecular genetics, virology, pharmacology and organic chemistry are those well-known and commonly employed in the art.

Standard techniques are used for biochemical and/or biological manipulations. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the terms “analog,” “analogue,” or “derivative” are meant to refer to a chemical compound or molecule prepared from another compound or molecule by one or more chemical reactions. As such, an analog can be a structure similar to, or based on, the structure of any small molecule inhibitor described herein, and/or may have a similar or dissimilar metabolic behavior.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, the term “pharmaceutical composition” or “composition” refers to a mixture of at least one compound useful within the invention with a pharmaceutically acceptable carrier. The pharmaceutical composition facilitates administration of the compound to a subject.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof. Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

The terms “pharmaceutically effective amount” and “effective amount” refer to a nontoxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation. By “pharmaceutical formulation” it is further meant that the carrier, solvent, excipient(s) and/or salt must be compatible with the active ingredient of the formulation (e.g. a compound of the invention). It is understood by those of ordinary skill in this art that the terms “pharmaceutical formulation” and “pharmaceutical composition” are generally interchangeable, and they are so used for the purposes of this application.

As used herein, the term “prevent,” “prevention,” or “preventing” refers to any method to partially or completely prevent or delay the onset of one or more symptoms or features of a disease, disorder, and/or condition. Prevention is causing the clinical symptoms of the disease state not to develop, i.e., inhibiting the onset of disease, in a subject that may be exposed to or predisposed to the disease state, but does not yet experience or display symptoms of the disease state. Prevention may be administered to a subject who does not exhibit signs of a disease, disorder, and/or condition.

As used herein, the term “subject,” “patient” or “individual” to which administration is contemplated includes, but is not limited to, humans (i.e., a male or female of any age group, e.g., a pediatric subject (e g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

As used herein, the term “therapeutically effective amount” is an amount of a compound of the invention, that when administered to a patient, treats, minimizes and/or ameliorates a symptom of the disease or disorder. The amount of a compound of the invention that constitutes a “therapeutically effective amount” will vary depending on the compound, the disease state and its severity, the age of the patient to be treated, and the like. The therapeutically effective amount can be determined routinely by one of ordinary skill in the art having regard to his own knowledge and to this disclosure.

The terms “treat,” “treating,” and “treatment,” refer to therapeutic or preventative measures described herein. The methods of “treatment” employ administration to a subject, in need of such treatment, a composition of the present invention, for example, a subject afflicted a disease or disorder, or a subject who ultimately may acquire such a disease or disorder, in order to prevent, cure, delay, reduce the severity of, or ameliorate one or more symptoms of the disorder or recurring disorder, or in order to prolong the survival of a subject beyond that expected in the absence of such treatment.

As used herein, the following abbreviations are presented: ACV, Aciclovir; CDV, Cidofovir; CMV, cytomegalovirus; ds, double-stranded; EBV, Epstein Barr virus; FOS, Foscarnet; hCMV, human cytomegalovirus; GCV, Ganciclovir; HDAC, histone deacetylase; HHV-5, human herpesvirus 5; HIV, human immunodeficiency virus; HSV, herpes simplex virus; mCMV, mouse CMV; SID, sec-butylisopropylacetamide (or 2-isopropyl-3-methylpentanamide); SIN, Sindbis virus; SPD, sec-butylpropylacetamide (or 3-methyl-2-propylpentanamide); ss, single-stranded; VACV, Valaciclovir; VCD, valnoctamide; VGCV, Valganciclovir; VPA, valproate; VPD/VPM, valpromide; VSV, vesicular stomatitis virus; VZV, varicella-zoster virus; TED, tert-butylethylacetamide (or 2-ethyl-3,3-dimethyl butanamide).

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds and Compositions

Compounds useful within the present invention comprise, in a non-limiting manner: valpromide (VPD or VPM; 2-propylpentanamide):

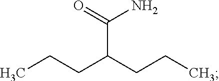

valnoctamide (VCD; 2-ethyl-3-methylpentanamide)

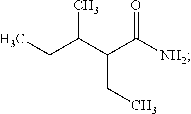

tert-butylethylacetamide (TED;
2-ethyl-3,3-dimethylbutanamide)

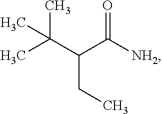

and a compound of formula (I):

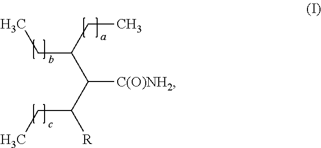

wherein:
a is 0, 1, 2, 3 or 4; b is 1, 2 or 3; c is 1, 2 or 3; and R is selected from the group consisting of H and C₁-C₄ alkyl, or solvates, salts, enantiomers, diastereoisomers or any mixtures thereof.

In certain embodiments, a is 0. In other embodiments, R is H. In yet other embodiments, R is 0 and a is 0. In yet other embodiments, a is 0, b is 1 and c is 1.

In certain embodiments, R is H, a is 0, b is 1 and c is 1, and the compound is the compound of structure (II) or solvates, salts, enantiomers, diastereoisomers or any mixtures thereof:

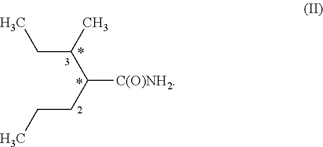

In certain embodiments, R is CH₃, a is 0, b is 0 and c is 1, and the compound is the compound of structure (III) or solvates, salts, enantiomers, diastereoisomers or any mixtures thereof:

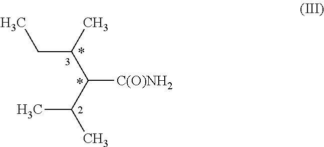

In certain embodiments, compound (II) is a diastereoisomer selected from the group consisting of (2R,3R), (2R,3S), (2S,3R) and (2S,3S), or any mixtures thereof.

In certain embodiments, compound (III) is a diastereoisomer selected from the group consisting of (2R,3R), (2R,3S), (2S,3R) and (2S,3S), or any mixtures thereof.

In certain embodiments, compound (I), (II) or (III) is in an enantiomerically and/or diastereoisomerically pure form. In other embodiments, compound (I), (II) or (III) is in an enantiomeric and/or diastereoisomeric mixture.

The compounds of the invention may possess one or more stereocenters, and each stereocenter may exist independently in either the (R) or (S) configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or mixtures thereof, or in the case where two or more chiral center are present, all diastereomers or mixtures thereof.

In certain embodiments, the compounds of the invention exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed invention. The compounds of the invention may contain any of the substituents, or combinations of substituents, provided herein.

The present invention includes pharmaceutical compositions comprising any compound useful within the invention. The pharmaceutical compositions described herein may be prepared by any method known or hereafter developed in the art of pharmacology. In general, such preparatory methods include bringing the active ingredient into association with a carrier or one or more other accessory ingredients, and then, if necessary or desirable, shaping or packaging the product into a desired single- or multi-dose unit.

### Salts

The compounds described herein may form salts with acids and/or bases, and such salts are included in the present invention. In certain embodiments, the salts are pharmaceutically acceptable salts. The term “salts” embraces addition salts of free acids and/or basis that are useful within the methods of the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (also known as N-methylglucamine) and procaine. All of these salts may be prepared from the corresponding compound by reacting, for example, the appropriate acid or base with the compound.

### Combination Therapies

In certain embodiments, the compounds of the invention are useful in the methods of present invention when used concurrently with at least one additional compound useful for treating a viral infection in a mammal.

Non-limiting examples of additional compound useful for treating a viral infection in a mammal are: Ganciclovir (GCV) [9-(1,3-dihydroxy-2-propoxymethyl)guanine]; acyclic nucleoside analog of 2′-deoxyguanosine and inhibits the viral DNA polymerase; Valganciclovir (VGCV): prodrug of GCV, with higher oral bioavailability; Foscarnet (FOS): pyrophosphate analog that inhibits the viral DNA polymerase; Cidofovir (CDV) [(S)-1-(3-hydroxy-2-phosphonylmethoxypropyl)cytosine]: acyclic nucleoside phosphonate analog of dCMP and acts by inhibiting the viral DNA polymerase; Fomivirsen (5′-GCGTTTGCTCTTCTTCTTGCG-3′; SEQ ID NO:1): antisense oligonucleotide against hCMV Major Immediate Early gene locus; Aciclovir (ACV) [9-(2-hydroxyethoxymethyl)guanine): analog of 2′-deoxyguanosine and inhibits viral DNA polymerase; Valaciclovir (VACV): prodrug of ACV, with improved oral bioavailability.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to elsewhere herein may be applied to experimental data to generate a corresponding graph to aid in assessing the effects of the drug combination. The corresponding graphs associated with the equations referred to elsewhere herein are the concentration-effect curve, isobologram curve and combination index curve, respectively.

### Methods

The present invention includes methods of treating and/or preventing a Herpesviridae viral infection in a mammal in a subject in need thereof. The method comprises administering to the subject a therapeutically effective amount of a compound contemplated in the invention, or any solvate, salt, enantiomer, diastereoisomer or any mixtures thereof.

In certain embodiments, the compound is part of a pharmaceutical composition or formulation further comprising at least a pharmaceutically acceptable carrier.

In certain embodiments, the administration of the compound has no significant anticonvulsant effect in the subject. In other embodiments, the administration of the compound has no significant mood stabilizing and/or modifying effect (including antimigraine effect) in the subject.

In certain embodiments, the subject is further administered one or more additional agents useful for treating and/or preventing the viral infection. In certain embodiments, the compound and the one or more additional agents are co-administered to the subject. In other embodiments, the compound and the one or more additional agents are coformulated.

In certain embodiments, the compound is administered to the subject by at least one route selected from the group consisting of oral, nasal, inhalational, topical, buccal, rectal, pleural, peritoneal, intra-peritoneal, vaginal, intramuscular, subcutaneous, transdermal, epidural, intratracheal, otic, intraocular, intrathecal, intra-amniotic, intra-umbilical cord, and intravenous routes. In other embodiments, the therapeutically effective amount of the compound ranges from about 0.001 mg/day to about 10,000 mg/day. In yet other embodiments, the compound is administered every day. In other embodiments, the compound is administered six days per week with one rest (no-administration) day. In yet other embodiments, the compound is administered five days per week with two rest days. In yet other embodiments, the compound is administered once a day four days per week with three rest days. In yet other embodiments, the compound is administered once a day three days per week with four rest days. In yet other embodiments, the compound is administered once a day per week with six rest days. The administration days may be consecutive or alternated with one or more rest days.

In certain embodiments, the subject is a mammal. In other embodiments, the subject is human. In yet other embodiments, the subject is pregnant. In yet other embodiments, the subject is neonatal. In yet other embodiments, the subject is an unborn fetus.

### Administration/Dosage/Formulations

The regimen of administration may affect what constitutes an effective amount. The therapeutic formulations may be administered to the subject either prior to or after the onset of a disease or disorder contemplated in the invention. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated in the invention. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the invention. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/day. The pharmaceutical compositions useful for practicing the invention may be administered to deliver a dose of from 1 ng/kg/day and 10,000 mg/kg/day. In other embodiments, the therapeutically effective amount of the compound ranges from about 10 μg/kg/day to about 1,000 mg/kg/day. In yet other embodiments, the therapeutically effective amount of the compound is about 100 mg/kg/day.

One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated in the invention.

In certain embodiments, the compositions of the invention are formulated using one or more pharmaceutically acceptable excipients or carriers. In other embodiments, the pharmaceutical compositions of the invention comprise a therapeutically effective amount of a compound of the invention and a pharmaceutically acceptable carrier. In yet other embodiments, the compound of the invention is the only biologically active agent (i.e., capable of treating a viral infection) in the composition. In yet other embodiments, the compound of the invention is the only biologically active agent (i.e., capable of treating a viral infection) in therapeutically effective amounts in the composition.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

In certain embodiments, the compositions of the invention are administered after a standard course of chemotherapy has been completed. In other embodiments the compositions of the invention are administered as a maintenance and/or preventive treatment. In certain embodiments, the maintenance and/or preventive treatments of the compound ranges from about 1 mg/kg/day to about 1,000 mg/kg/day. In yet other embodiments, the therapeutically effective amount of the compound is about 10-500 mg/kg/day. In yet other embodiments, the maintenance and/or preventive treatments are administered once per day and every day. In yet other embodiments, the maintenance and/or preventive treatments are administered once a day, six days per week with one rest day therein. In yet other embodiments, the maintenance and/or preventive treatments are administered once a day, five days per week with two rest days. In yet other embodiments, the maintenance and/or preventive treatments are administered once a day, four days per week with three rest days. In yet other embodiments, the maintenance and/or preventive treatments are administered once a day, three days per week with four rest days. The administration days may be consecutive or alternated with one or more rest days.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments there between.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated in the invention.

Formulations may be employed in admixtures with conventional excipients, i.e., pharmaceutically acceptable organic or inorganic carrier substances suitable for intra-peritoneal, oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents.

Routes of administration of any of the compositions of the invention include intra-peritoneal, oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-peritoneal, intra-arterial, intravenous, intrabronchial, inhalation, intra-amniotic, intra-umbilical cord and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY· White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation”. For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) melt.

The present invention also includes a multi-layer tablet comprising a layer providing for the delayed release of one or more compounds of the invention, and a further layer providing for the immediate release of a medication for treatment of a disease or disorder contemplated in the invention. Using a wax/pH-sensitive polymer mix, a gastric insoluble composition may be obtained in which the active ingredient is entrapped, ensuring its delayed release.

Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intra-aminiotic, intra-umbilical cord, and intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds useful within the methods of the invention may be administered in the form of microparticles, for example by injection, or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, or about 1 minute and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, or about 1 minute and any and all whole or partial increments thereof after drug administration.

### Dosing

The therapeutically effective amount or dose of a compound of the present invention depends on the age and weight of the patient, the current medical condition of the patient and the progression of a disease or disorder contemplated in the invention. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 5 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance and/or preventive dose is administered if necessary. Subsequently, the dosage or the frequency of administration, or both, is reduced, as a function of the disease or disorder, to a level at which the improved disease is retained. In certain embodiments, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 5 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Example 1:

### A. Materials and Methods

### Cells

NIH/3T3 (CRL-1658) and Vero (CCL-81) cells were purchased from the American Type Culture Collection (ATCC) (Manassas, Va.), normal human dermal fibroblasts were obtained from Cambrex (Walkersville, Md.), Neuro-2a (CCL-131) were provided by A. Bordey (Yale University, New Haven, Conn.), and U-373 MG cells were a gift from R. Matthews (Syracuse, Conn.). Vero cells were grown and maintained in Eagle's Minimum Essential Medium (MEM) supplemented with 10% fetal bovine serum (FBS) and 1% pen/strep (Invitrogen, Carlsbad, Califo.). All the other cell lines were maintained in Dulbecco's modified Eagle's essential medium (DMEM) supplemented with 10% FBS and 1% pen/strep. Primary cultures of mouse glia were established using whole brain tissue harvested from P5 mice and maintained in DMEM (van den Pol, et al., 1999, J. Neurosc. 10948-10965). All cultures were kept in a humified atmosphere containing 5% CO₂ at 37° C.

### Viruses

A brief description of each virus used is given below.

mCMV-GFP. Recombinant mCMV (MC.55) expressing EGFP was derived from the K181 strain. The expression cassette containing the EGFP gene controlled by the human elongation factor 1 alpha (EF1-alpha) promoter was inserted into the immediate early gene (IE-2) site. NIH/3T3 cells were used for viral propagation and plaque assay (van den Pol, et al., 1999, J. Neurosc. 10948-10965).

hCMV-GFP. Recombinant hCMV expressing EGFP under the control of the EF1-alpha promoter was kindly provided by J.Vieira (University of Washington, Seattle). The gene coding for EGFP was inserted between US9 and US10 of the human CMV genome, a site that appears to tolerate alterations without affecting viral replication. EGFP expression and replication capability were tested on normal human fibroblasts and U-373 human glioblastoma cells (J. Vieira, et al., 1998, J. Virol. 72:8158-8165; Jarvis, et al., 1999, J. Virol. 73:4552-4560).

VSV-GFP. A recombinant variant of the Indiana strain of VSV containing a GFP-VSV G fusion protein as an extra gene downstream of the native VSV G protein gene, kindly provided by J. K. Rose and K. Dalton (Yale University, New Haven, Conn.), was used in the present study. The virus was maintained on monkey kidney epithelial cells (Vero). Vero cells were used for viral propagation and plaque assay (van den Pol, et al., 2002, J. Virol. 76:1309-1327).

SIN-GFP. A recombinant SIN (an alphavirus) expressing GFP was a gift from J. M. Hardwick (Johns Hopkins University, Baltimore, Md.). A construct consisting of a duplicated copy of the viral sub-genomic promoter and a BstEII cloning site was inserted into the 3′ regulatory region of SIN genome plasmid. The cloning site can be universally used for the expression of genes of interest, in this case GFP. Vero cells were used for virus maintenance and plaque assay (Hardwick & Levine, 2000, Methods Enzymol. 322:492-508).

All the viruses listed express EGFP as a reporter for cell infection. Green fluorescence was employed to visualize infected cells and plaques. Plaque titers were determined by a standard plaque assay technique using cell monolayers and a carboxy-methyl-cellulose (CMC)-based viscous overlay for mCMV-GFP and hCMV-GFP (35) or a 0.5% agar overlay for VSV-GFP and SIN-GFP. Viral stocks were stored in aliquots at −80° C. For each experiment, a new aliquot of virus was thawed and used.

### Chemicals

Valpromide (catalog no. V3640), valnoctamide (catalog no. V4765), valproate (catalog no. S0930000), ivermectin (catalog no. 188998), and heparan sulfate sodium salt (catalog no. H7640) were purchased from Sigma-Aldrich (St. Louis, Mo.). Valproate and heparan sulfate were dissolved in water to give a stock solution of 1 M and 1 mg/mL, respectively. Valpromide, valnoctamide, sec-butylpropylacetamide, sec-butylisopropylacetamide, tert-butylethylacetamide, and ivermectin were dissolved in dimethylsulfoxide (DMSO) to yield a stock solution of 1 M (valpromide, valnoctamide, sec-butylpropylacetamide, sec-butylisopropylacetamide, tert-butylethylacetamide), and 100 mM (ivermectin). Ivermectin was used at 1 μM, a concentration shown effective against Chikungunya virus and other alphaviruses.

### Quantification of Infection

Cells (NIH/3T3, Neuro-2a, mouse glia, Vero, normal human dermal fibroblasts, and U-373) were plated at a density of 40,000 cells per well in 48-well dishes and incubated overnight before medium (0.2 mL per well) was replaced for pre-treatment with VPA, VPD, VCD, ivermectin, or vehicle at the specified concentrations. After 4, 24, or 72 hours of drug exposure, cells were inoculated with virus. Both high and low MOI were used for hCMV-GFP and mCMV-GFP; precisely, 0.1 (hCMV, mCMV) and 0.4 (mCMV) as low MOI, and 1 (mCMV), and 4 (mCMV and hCMV) as high MOI. The other tested viruses were used at low MOI, i.e. 0.001.

In some experiments, cultures were inoculated with virus and simultaneously exposed to the drugs for 2 hours (‘short drug exposure’ experiment) or 48 hours (time-of-drug addition' experiment). In ‘time-of-drug addition’ experiment, administration of the compounds was performed not only at the same time as viral challenge, but also at subsequent time-points (2 and 12 hours), in order to assess which step of the viral replicative cycle was affected by the drugs.

To test the effects of a short drug exposure (2 hours) on viral production and release, a viral yield reduction assay was performed. NIH/3T3 cells were infected with mCMV-GFP (MOI 0.1) and simultaneously treated with 100 μM compounds. After 2 hours of incubation at 37° C., to allow viral adsorption, cultures were rinsed twice with PBS and replenished with drug-free media. 72 hpi cell culture supernatants were collected and plaque-titrated on NIH/3T3 monolayers.

To investigate the effects on the immediate early phase of viral replication, NIH/3T3 cells exposed to VPD (1 mM) for 24 hours, were transfected with a CMV promoter (IE1/IE2)-driven reporter plasmid (pCMV-tdTomato) expressing the red fluorescent protein tdTomato.

For assessing the potential virucidal activity of the tested compounds on free virions (virucidal activity assay), VPD, VCD, or vehicle at 100 μm were added to an undiluted stock of mCMV-GFP, and this compounds/virus mixture was incubated at either 4° C. or 37° C. for 2 hours. After incubation, the solution was diluted with culture medium to reduce the amount of drug to a concentration identified as ineffective, i.e., 10 nM, and the residual mCMV-GFP infectivity was then plaque-titrated on NIH/3T3 cells.

To evaluate the drug effects on viral binding and fusion to the cell membrane, cultures were first incubated at 4° C. (to allow binding but not fusion) for 2 hours, and then at 37° C. (to allow fusion and subsequent steps of the viral replicative cycle) (Chan & Yurochko, 2014, Methods Mol. Biol. 1119:113-121). In the binding experiments, NIH/3T3 cells were simultaneously exposed to drugs (VPD, VCD, or HS) or vehicle at 100 μM and mCMV-GFP (MOI 0.1), and incubated at 4° C. for 2 hours. HS was used as positive control since its inhibitory activity on CMV attachment to cell surface. Cultures were then rinsed twice with PBS, to remove the unbound virions and the compounds, replenished with fresh drug-free media, and incubated at 37° C. for 2 hours, to allow viral fusion and adsorption. Finally, cells were washed twice with PBS and overlaid with a viscous solution containing DMEM (75%) and CMC (25%) (Zurbach, et al., 2014, Virol. J. 11:71-79). In the fusion experiments, cultures plated in plain media were inoculated with mCMV-GFP (MOI 0.1) and incubated at 4° C. for 2 hours. NIH/3T3 were then washed twice, exposed to the tested compounds (100 μM), and incubated at 37° C. for 2 hours before being rinsed twice and overlaid with a CMC-based solution. Infectivity was assessed at 48 hpi for both experiments.

Infected cells were identified as GFP-positive cells using an Olympus IX71 fluorescence microscope (Olympus Optical, Tokyo, Japan). The total number of fluorescent cells per well in each condition was counted. Each condition was tested at least in triplicate, and the whole experiment repeated twice. The microscope was connected to a SPOT RT digital camera (Diagnostic Instruments, Sterling Heights, Mich.) interfaced with an Apple Macintosh computer. Conditions (exposure time and gain) were kept consistent between images. The contrast and color of collected images were corrected using Adobe Photoshop.

Cytotoxicity assay

An ethidium homodimer assay (Molecular Probes, Eugene, Oreg.) was used to label dead cells according to the manufacturer's instructions. Briefly, NIH/3T3 cells (9×10⁴ per well) were seeded in a 48-well plate and treated with VPD or vehicle for 24 hours before mCMV-GFP inoculation (MOI 0.4). 72 hours after viral challenge, cells were washed twice and EthD-1 was added at a final concentration of 4 μM in DMEM. After 20 minutes of incubation at 37° C., the total number of dead cells per well was counted based on red fluorescence of nuclei. Each condition was tested in quadruplicate, and each experiment was repeated twice.

### Plaque Size Assay

Plaque size assay was used to assess the effect of the drugs on viral propagation. Briefly, semiconfluent NIH/3T3 cells in 12-well plates were inoculated using mCMV-GFP. After 2 h-incubation at 37° C. to allow viral adsorption, inoculum was removed and cultures were washed three times with PBS before the addition of a viscous overlay solution containing VPD, VCD, or vehicle at the specified concentrations diluted in DMEM (75%) and CMC (25%) (Zurbach, et al., 2014, Virol. J. 11:71-79). Five days later, the relative size of viral plaques was measured (n=60 plaques/condition), as described in Wollmann, et al., 2015, J. Virol. 89:6711-6724). Each condition was tested at least in triplicate, and the experiment repeated twice. All measurements were performed at the same time with similar exposure time and gain.

### Animal Procedures

Male and female Balb/c strain mice (6-8 weeks of age) from Taconic Biosciences Inc (Hudson, N.Y.) were maintained on a 12:12-h light cycle under constant temperature (22±2° C.) and humidity (55 ±5%), with access to food and water ad libitum. One to two females were cohabited with a male of the same strain for at least 1 week to ensure fertilization. When advanced pregnancy was seen, each pregnant female was caged singularly and checked for delivery twice daily, at 8:30 AM and 6:30 PM. Newborns were inoculated intraperitoneally (i.p.) with 750 plaque-forming unit (PFU) of mCMV-EGFP in 50 μL of media on the DOB, within 14 hours of delivery. The DOB was considered PND 0. Control animals received 50 μL of media i.p. Infected pups were randomly assigned to receive VPD, VCD, or vehicle (DMSO), via subcutaneous (s.c.) injections, once a day, at a dose of 1.4 mg/mL in 20 μL of saline (˜30 μg), starting after virus inoculation from PND 1 to PND 21. Control pups received a similar amount of drug-free saline. Mice were monitored daily for survival until PND 49. Additionally, on each day from PND 0 to 22, without knowledge of the treatment group, pups were weighed to the nearest 0.01 g and their body and tail lengths were measured. Hair growth, status of eyelid and pinnae detachment, and incisor eruption, as compared to adult mice, were also recorded, as described in Scattoni, et al., 2008, PLoS One 3, 10.1371/journal.pone.0003067. Briefly, these somatic variables were rated semi-quantitatively in the following way: 0=no occurrence of the condition, 1=slight/uncertain condition, 2=incomplete condition, and 3=a complete adult-like condition.

For detection of infectious viral load in organs, some of the control and experimental mice were sacrificed on PND 12, after receiving saline/treatment from PND 1 to PND 10. Designated mice were transcardially perfused with PBS, to wash out free virus, and tissue samples were collected under sterile conditions from liver and lungs, two organs markedly involved in severe perinatal infection in humans. Tissues were mechanically homogenized in PBS using a microcentrifuge tube tissue grinder. Part of the resulting tissue suspension was plated onto NIH/3T3 monolayers and viral titer was assessed using the plaque assay technique (Zurbach, et al., 2014, Virol. J. 11:71-79).

### Statistical Analysis

In the experiments for quantification of infection and cytotoxicity, and in the plaque reduction assay, statistical significance was determined using one-way ANOVA, followed by post-hoc analysis (Bonferroni's test). Data are presented as percentage of infected or dead cells, and as viral titers, in drug versus vehicle, as mean±SEM of two independent experiments; each independent experiment consisted of three or four cultures; p-values refer to a comparison of drug to control (vehicle). For markers of somatic development, a mixed-model ANOVA with PND as the Repeated Measures factor was used, followed by Newman Keuls test only if a significant F-value was determined.

Analysis was performed with GraphPad Prism 6.0 and SPSS Statistics 21, with significance set at p<0.05. Survival studies and assessment of somatic development parameters were performed blindly with respect to the experimental group.

### B. Discussion

As demonstrated herein, the present studies comprise testing whether VPD causes a reduced enhancement of CMV infection compared with VPA.

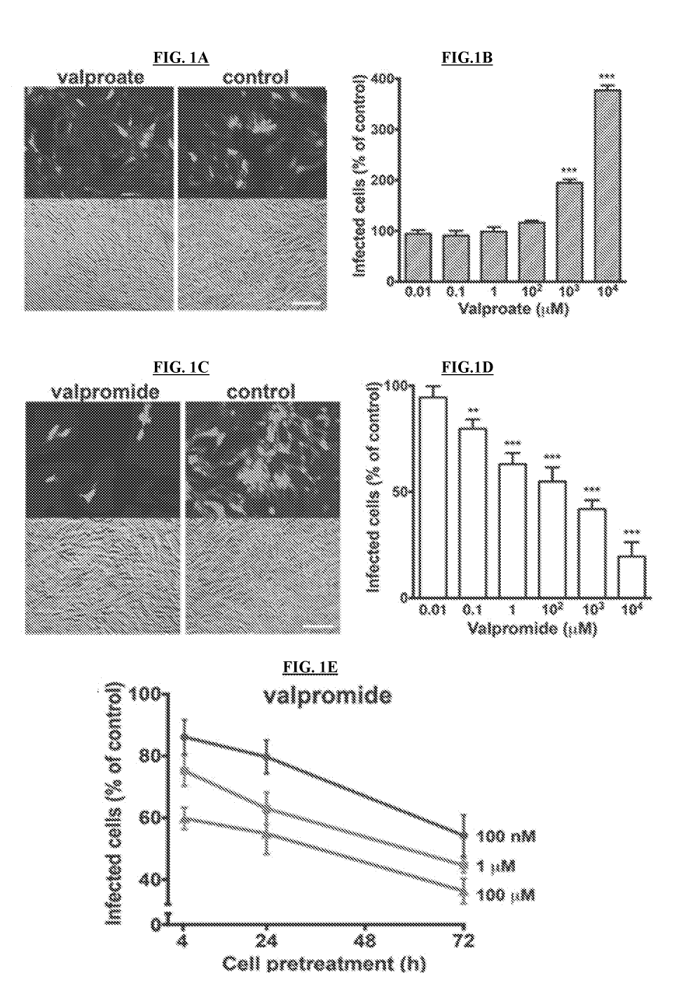

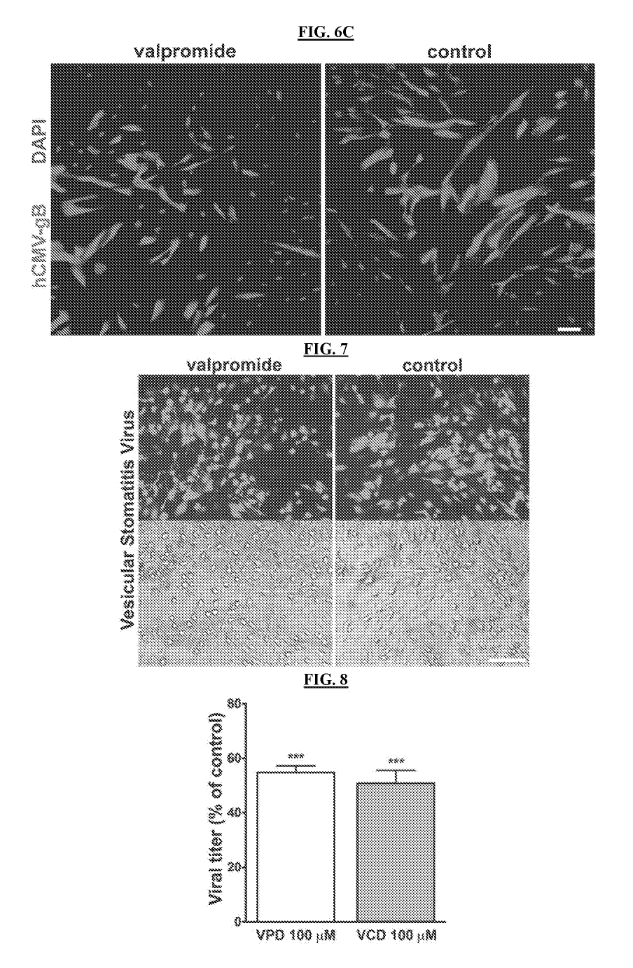

VPA increased infection by mouse CMV (mCMV) (FIGS. 1A-1B). VPD showed a robust dose-dependent inhibitory effect, even at low drug concentrations (FIGS. 1C-1E). Inhibition was confirmed at high virus titer and in multiple cell types (FIGS. 5A-5D). VPD-mediated CMV inhibition increased cell survival (FIGS. 5E-5F) and prolonged pre-virus drug exposure showed efficacy at nM concentrations (FIG. 1E). VPD inhibited human CMV infection, independent of virus titer or cell type (FIGS. 6A-6B). Immunocytochemistry for the hCMV glycoprotein B showed fewer CMV immunoreactive cells in cultures treated with VPD than in controls (FIG. 6C).

The drug-mediated inhibition of CMV raised the question of whether the VPD antiviral effect was universal for different types of virus and might act to enhance an innate immune block of infection. To address this question, we tested non-related viruses including vesicular stomatitis virus (VSV) and Sindbis virus (SIN). VPD did not inhibit VSV (FIG. 7) or SIN (107%±3% compared to control), suggesting that VPD antiviral actions were specific for CMV over these other viruses. Ivermectin, a compound with anti-epileptic properties and a strong anti-parasitic activity known to attenuate alphavirus infection, was also tested. Ivermectin had no effect on CMV (99%±9% compared to control), demonstrating that the anti-CMV effect was specific for VPD and VCD.

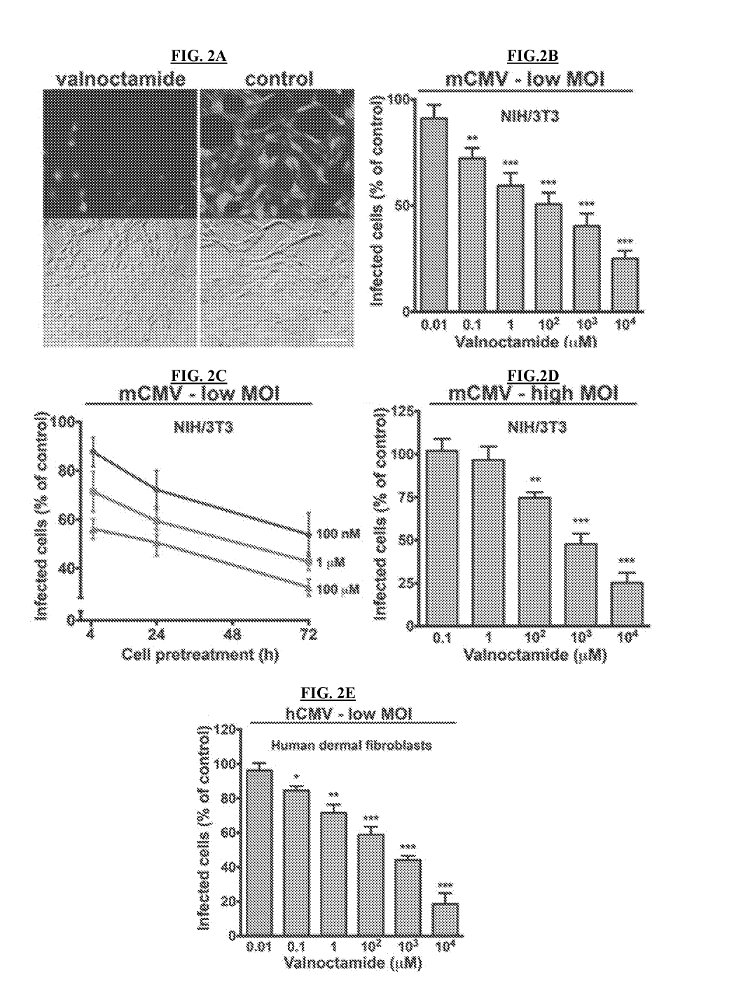

The initial experiments showed a strong and specific inhibitory effect of VPD on CMV. However, VPD safety in animal models of teratogenesis may not apply to humans in whom VPD is rapidly metabolized (>80%) to the pro-CMV and teratogenic VPA. Thus, valnoctamide (VCD), that also lacks HDAC inhibitory activity, was also tested. Unlike VPD, VCD shows little conversion to its corresponding free acid (valnoctic acid) in humans. VCD is used as a mild tranquilizer, and may attenuate acute mania. VCD inhibited both mouse (FIGS. 2A-2D) and human CMV (FIG. 2E), in a dose-dependent fashion, with inhibitory activity evident at low drug concentrations.

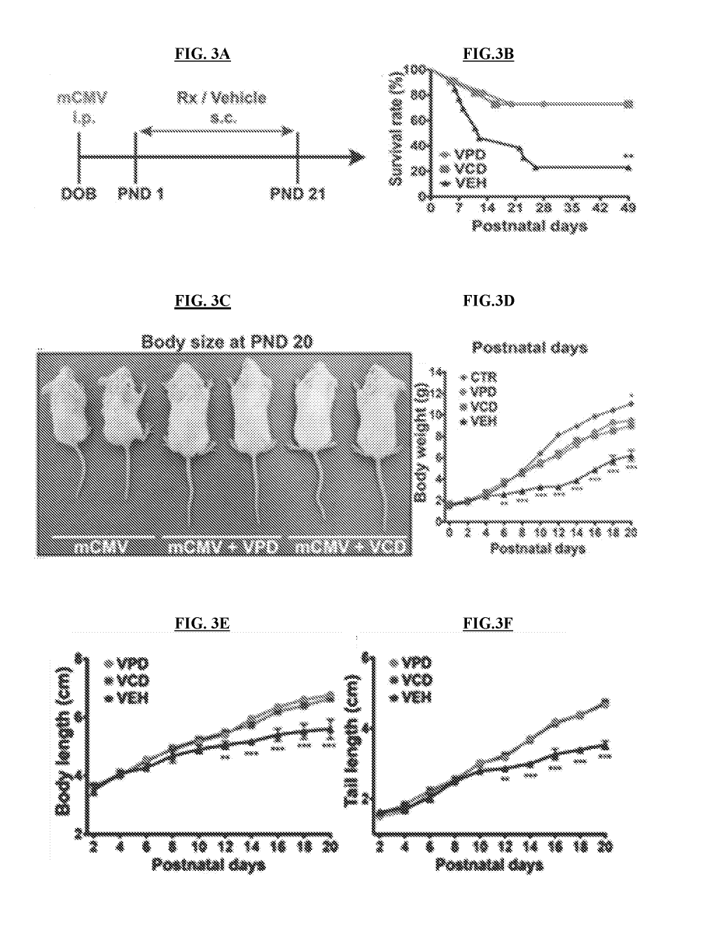

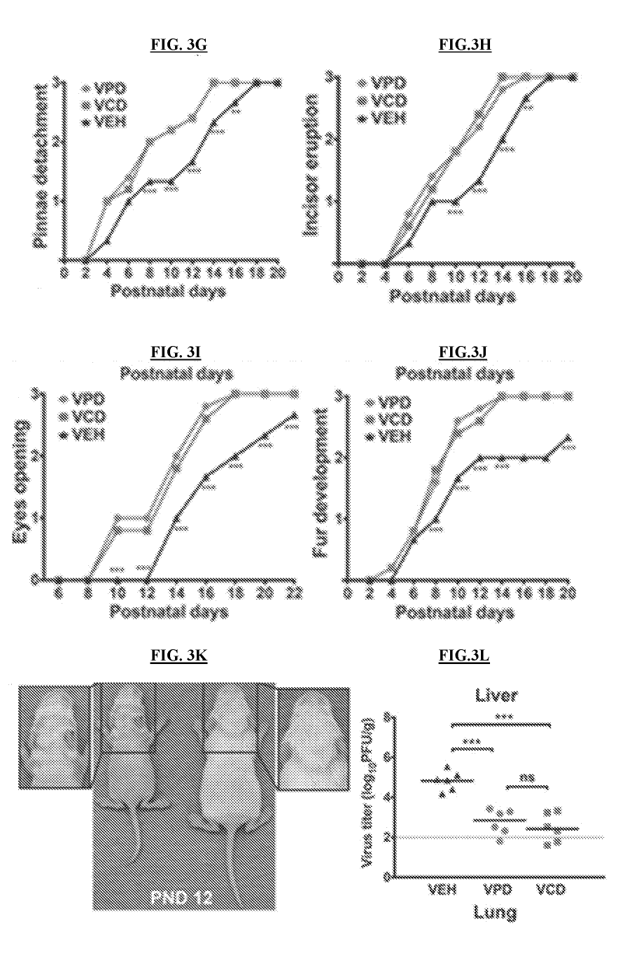

It was then assessed whether VPD or VCD suppresses CMV in vivo. Both drugs were assessed in a mouse model of severe perinatal mCMV infection (FIG. 3A). VPD and VCD both reduced the death rate by three-fold, and increased survival from 23% of mCMV infected pups to 72% (FIG. 3B). Infected newborns treated with VPD and VCD showed additional drug benefits. Treatment increased body growth after CMV infection. Infected mice weighed nearly 50% less than control mice at post-natal day 20, whereas VPD- or VCD-treated infected pups showed a body weight reduction of only 18% (FIGS. 3C-3D). Drug administration markedly improved multiple parameters of somatic development, including body and tail length, eyelid opening, and fur maturation (FIGS. 3E-3L). CMV load in liver and lungs was markedly decreased by both compounds (FIGS. 3M-3N). The drugs generated a significant (p<0.05) improvement in CMV-infected neonate health as early as 5 days after initiation of drug treatment.

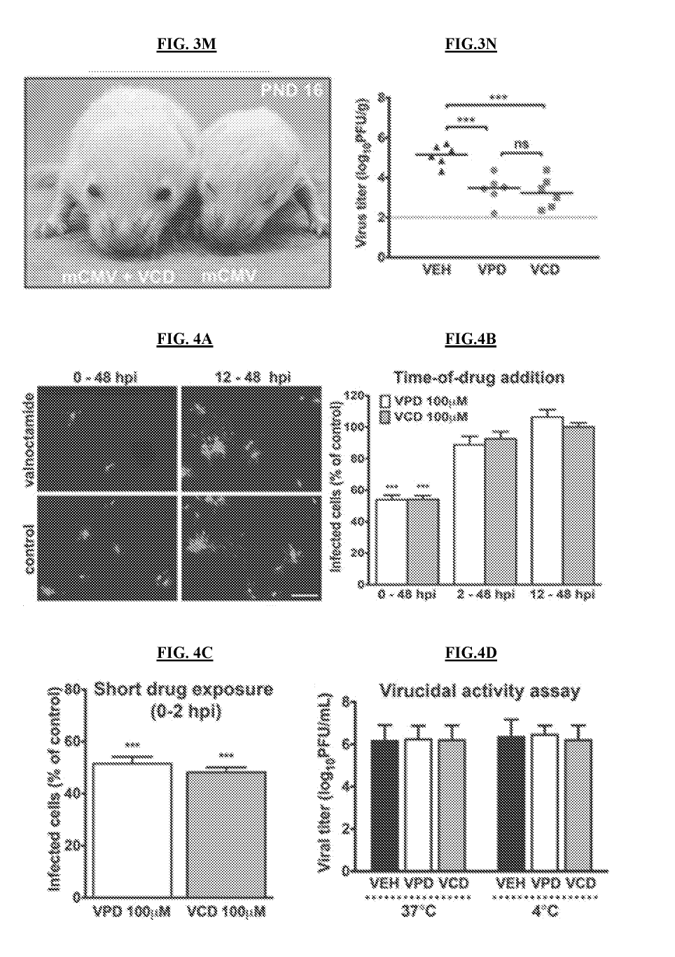

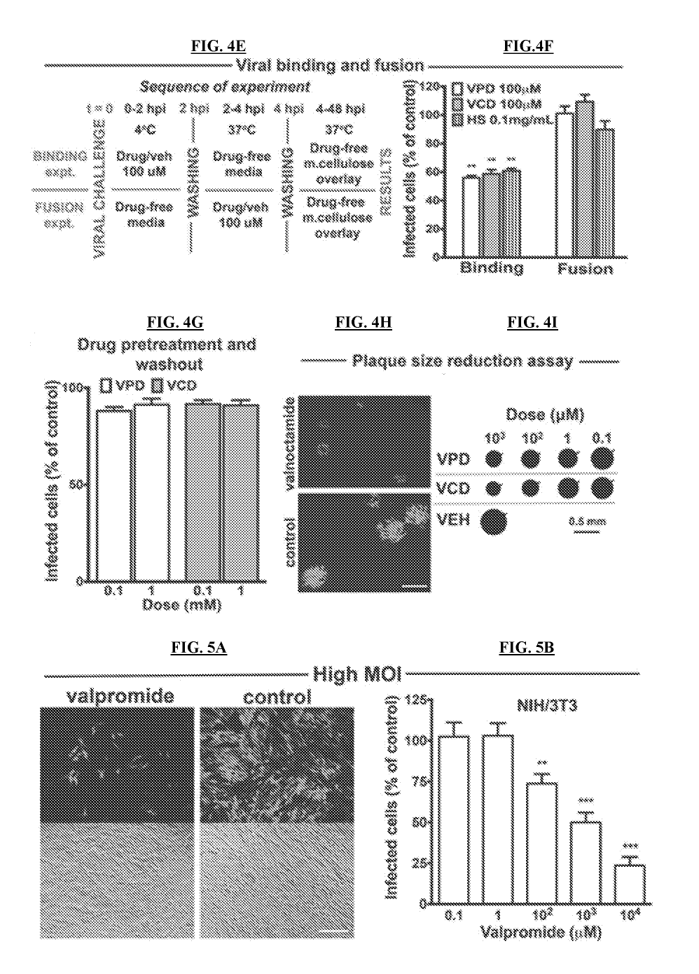

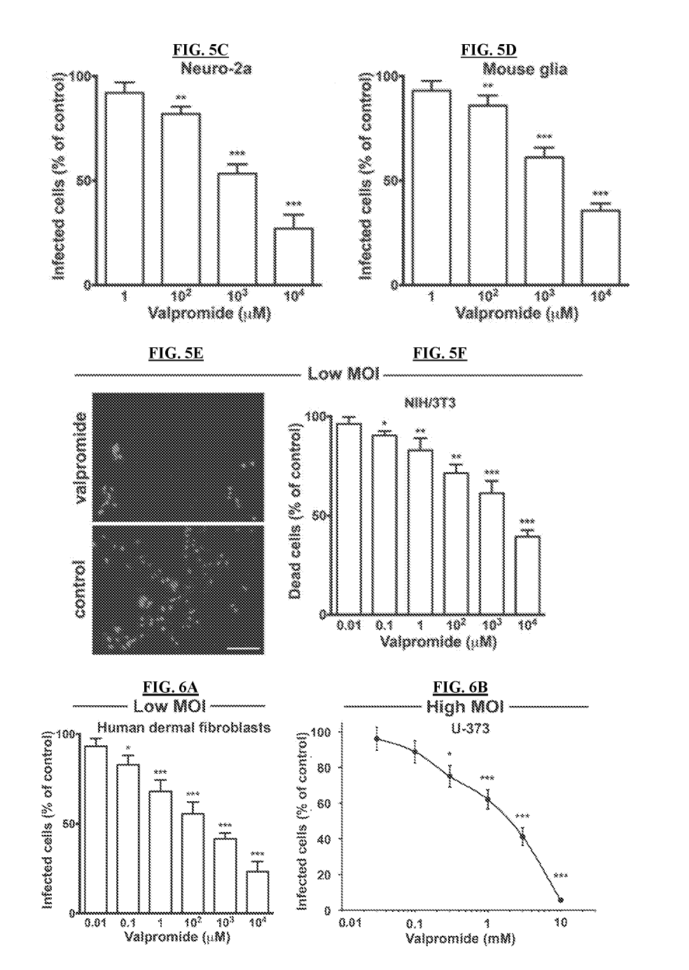

Despite being used for decades to treat neurological dysfunction, the mechanism(s) of action of VPD and VCD in the brain is still incompletely elucidated. VPA-mediated inhibition of HDAC enhances infection by hCMV. Both VPD and VCD lack this epigenetic function. To gain insight into the actions of VPD and VCD on CMV, the drugs at low concentration were added at different time-points in the course of mCMV infection (FIGS. 4A-4B). When the inhibitors were present from the time of viral challenge through 48 hours post infection (hpi) (0-48 hpi), the number of infected cells decreased to 50%; a similar CMV inhibition was also observed for brief drug exposure at the beginning of virus infection (0-2 hpi) (FIG. 4C). However, no inhibition was identified when compounds were added 2 to 12 hours after virus inoculation. A 2 hr drug exposure at the time of inoculation, followed by drug washout, yielded a strong CMV inhibition (FIG. 8).

These results suggest that the block of CMV mediated by VPD and VCD is exerted early in the infection process. The immediate early (IE)1/2 CMV promoter is active in the first few hours of CMV infection. In order to investigate whether the drugs acted on this promoter, a plasmid with CMV IE1/2 driving tdTomato expression was tested. No decrease in the number of red cells was identified in the presence of VPD compared to control (96%±4%) after plasmid transfection, suggesting VPD does not inhibit the CMV IE1/2 promoter. The possibility of a virucidal activity was tested by pre-incubating the compounds with an undiluted stock of mCMV prior to cell inoculation. No direct inactivation of virions was observed, as evidenced by no reduction in titer when the inhibitors were subsequently diluted below the effective concentrations before cell inoculation (FIG. 4D). Early stages of infection involve virus attachment to the cell surface (binding) and subsequent penetration (fusion). Without wishing to be limited by any theory, both drugs may inhibit viral binding (FIGS. 4E-4F). Drug/cell interaction analysis showed a reversible drug effect with loss of antiviral activity if compounds were removed before virus inoculation (FIG. 4G). Without wishing to be limited by any theory, a time-dependent increase in drug binding to its cellular targets could constitute one mechanism underlying the enhanced viral inhibition observed with prolonged cell pre-exposure to low drug concentrations (FIGS. 1E and 2C). VPD and VCD decreased spread of the CMV infection as shown by reduced plaque size in CMV infected cells (FIGS. 4H-4I).

Further, the following exemplary compounds were tested within the methods of the invention: sec-butylpropylacetamide (SPD; 3-methyl-2-propylpentanamide), sec-butylisopropylacetamide (SID; 2-isopropyl-3-methylpentanamide), and tert-butylethylacetamide (TED; 2-ethyl-3,3-dimethylbutanamide).

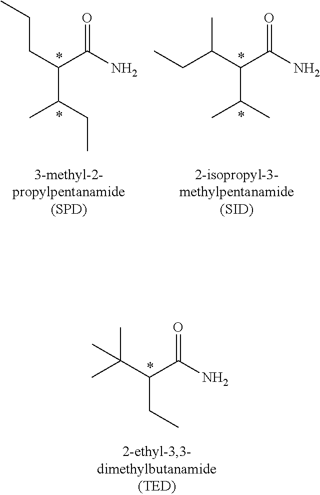

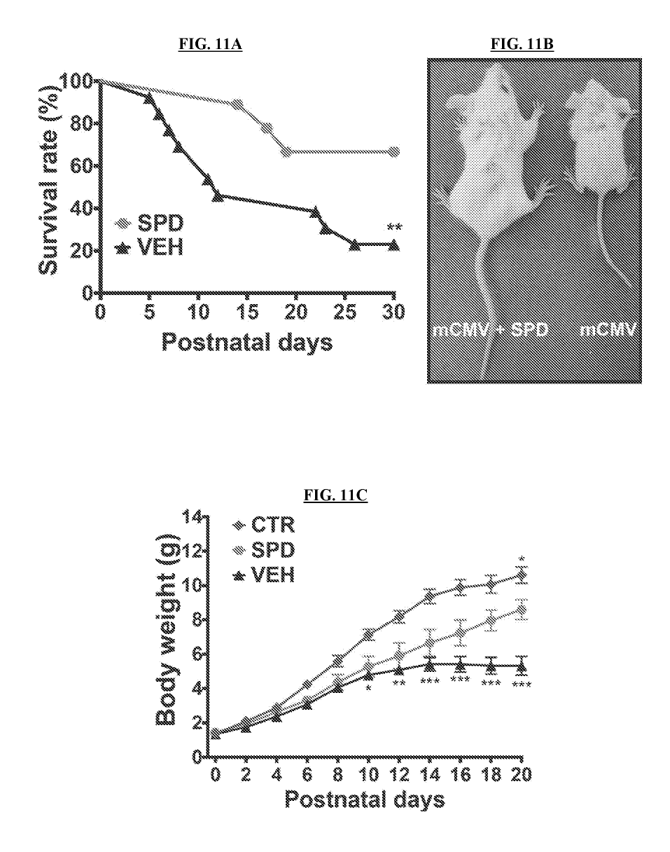

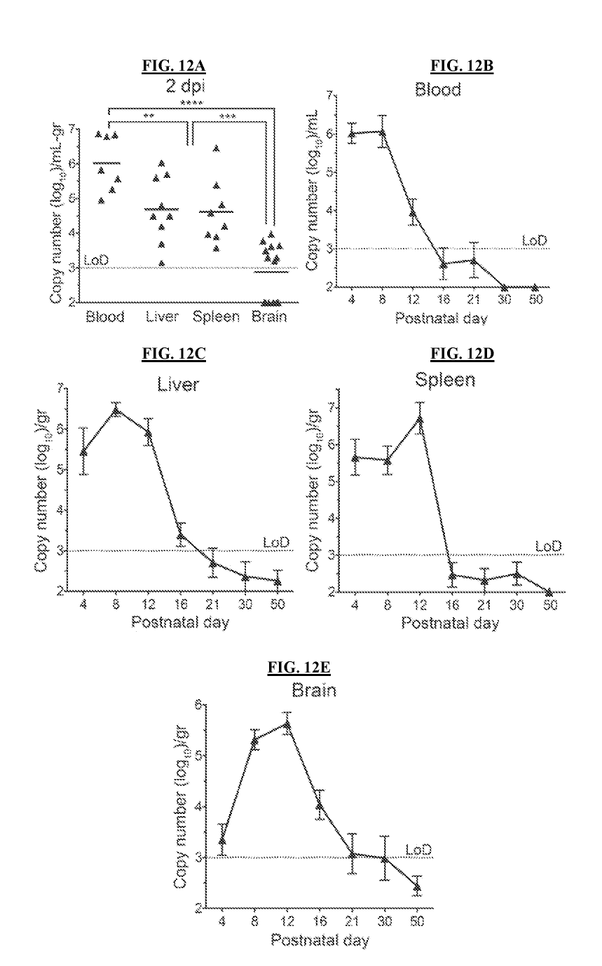

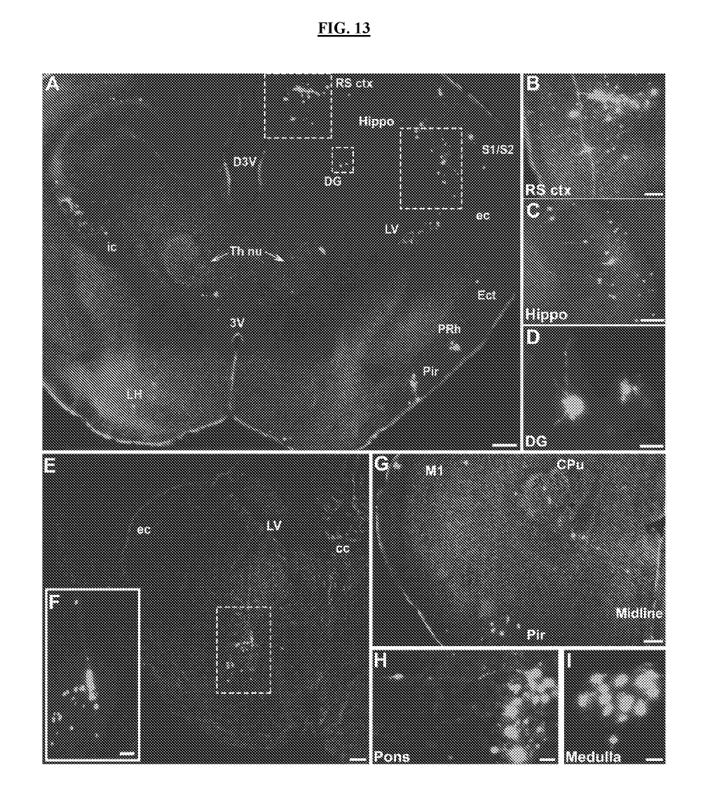

SPD was very effective in inhibiting CMV (FIG. 10). SPD is a one-carbon homologue of VCD. Similar to VCD, SPD possesses two stereogenic carbons in its structure and may exist as 4 distinct stereoisomers. SPD and its individual stereoisomers lack a teratogenic effect on developing animals. SPD in vitro was slightly more effective than either valpromide or valnoctamide at similar concentrations (FIG. 10). In the same animal model of severe in vivo perinatal infection employed for testing VPD and VCD and with the same schedule of administration, SPD showed similar efficacy to VPD and VCD (FIGS. 11A-11C). SPD increased survival of infected pups by three-fold (FIG. 11A), from 23% of mCMV infected untreated pups to 67%, and significantly improved post-natal body growth (FIGS. 11B-11C). SPD-treated newborns weighed only 20% less than control mice at post-natal day 20, whereas infected, not treated pups showed a 50% decrease in body weight.

### Example 2:

### A. Materials and Methods

Cell lines, Viruses, and Chemicals:

Normal human dermal fibroblasts (HDF) were obtained from Cambrex (Walkersville, Md.) and primary human fetal brain astrocytes were obtained from ScienceCell Research Laboratories (Carlsbad, Calif.). HDF cells were cultured in Dulbecco's modified Eagle's essential medium (DMEM) supplemented with 10% FBS and 1% pen/strep (Invitrogen, Carlsbad, Calif.). Human fetal astrocytes were grown in poly-L-lysine coated culture vessels and maintained in Astrocyte Medium (ScienceCell) supplemented with 2% FBS and 1% pen/strep. All cultures were kept in a humidified atmosphere containing 5% CO₂ at 37° C.

For in vitro experiments, a recombinant human CMV (hCMV, Toledo strain) expressing enhanced green fluorescent protein (EGFP) under the control of the EF1-alpha promoter (EGFP-hCMV) was employed (Jarvis, et al., 1999, J. Virol. 73:4552-4560). Normal human fibroblasts were used to test viral EGFP expression, replication capability, propagation and for determining viral titers by plaque assay (Jarvis, et al., 1999, J. Virol. 73:4552-4560).

CMV replication is species-specific, and to study CMV in vivo, a recombinant mouse CMV (mCMV) CMV (MC.55, K181 strain) that expressed enhanced GFP (van Den Pol, et al., 1999, J. Neurosci. 19:10948-10965) was used (Ornaghi, et al., 2016, Virology 499:121-135). NIH/3T3 cells (murine fibroblasts) were used for viral propagation and titering by plaque assay (van Den Pol, et al., 1999, J. Neurosci. 19:10948-10965).

Recombinant CMVs were provided by Dr. E. Mocarski (Emory University, Atlanta) and Dr. J. Vieira (University of Washington, Seattle).

Green fluorescence was employed to visualize infected cells and viral plaques. Viral titers were determined by standard plaque assay using 25% carboxy-methyl-cellulose (CMC) overlay (Zurbach, et al., 2014, Virol. J. 11:71). Viral stocks were stored in aliquots at −80° C. For each experiment, a new aliquot of virus was thawed and used.

Valnoctamide (catalog no. V4765) was purchased from Sigma-Aldrich (St. Louis, Mo.) as powder and dissolved in dimethylsulfoxide (DMSO) to yield a stock solution of 1 M.

### Quantification of Infection:

Effects of VCD on hCMV infection were assessed by viral infectivity assay and viral yield reduction assay. For the infectivity assay, human fetal astrocytes were seeded at a density of 40,000 cells per well in 48-well plates and incubated overnight before medium (0.2 mL per well) was replaced for pre-treatment with VCD or vehicle at 100 μM. After 1 h of drug exposure, cells were inoculated with hCMV (MOI 0.1) and incubated at 37° C. for 2 h to allow viral adsorption. Following incubation, cultures were washed twice with PBS and overlaid with a viscous solution containing VCD/vehicle at 100 μM in supplemented Astrocyte Medium (75%) and CMC (25%). GFP-positive cells were counted at 48 hours post-infection (hpi).

In the virus yield reduction assay, after viral adsorption, cells were washed twice with PBS and replenished with fresh medium containing the compounds to be tested. At 72 hpi medium was collected and titered by plaque assay using HDF monolayers to assess the drug-mediated inhibition of virus replication in human fetal brain astrocytes.

The total number of fluorescent cells/plaques per well in each condition were counted using an Olympus IX71 fluorescence microscope (Olympus Optical, Tokyo, Japan) connected to a SPOT RT digital camera (Diagnostic Instruments, Sterling Heights, Mich.) interfaced with an Apple Macintosh computer. Each condition was tested in triplicate, and the whole experiment repeated twice. Camera settings (exposure time and gain) were held constant between images. The contrast and color of collected images were optimized using Adobe Photoshop.

### Viral Entry Analysis and Quantitative Real-Time PCR Assay:

To evaluate the effects of VCD on hCMV attachment to human fetal astrocytes, pre-chilled cultures at 90% confluency in a 6-well plate were treated with VCD or vehicle (100 μM) for 1 h at 4° C., followed by infection with pre-cooled hCMV-GFP (MOI 0.1). After 2 h incubation at 4° C. fetal astrocytes were rinsed three times with cold PBS to remove unattached virions, then harvested by trypsinization for viral DNA quantification using a quantitative real-time PCR (qPCR) assay (Chan & Yurochko, 2014, Methods Mol. Biol. 1119:113-121). To assess hCMV internalization into fetal astrocytes, cultures plated in plain media were inoculated with hCMV-GFP (MOI 0.1) and incubated at 4° C. for 2 h. Cells were then washed three times to remove unbound viral particles, and exposed to VCD or vehicle at 100 μM for 2 h at 37° C. (to allow virus internalization) before being harvested by trypsinization for DNA quantification by qPCR.

DNA was extracted from cells using the QIAamp DNA mini kit (Qiagen, Germantown, Md.) and qPCR was performed using TaqMan assays (Life Technologies) (Gault, et al., 2001, J. Clin. Microbiol. 39:772-775; Fukui, et al., Antimicrob. Agents Chemother. 52:2420-2427) for hCMV UL132 (Pa03453400_s1) and human albumin (Hs99999922_s1) genes, as previously described (Ornaghi, et al., 2016, Virology 499:121-135). Samples from uninfected cells and without template served as negative controls. Samples were run in duplicate using a Bio-Rad iCycler-IQ instrument (Bio-Rad, Hercules, Calif.), and results were analyzed with iCycler software. The amount of viral DNA in each sample relative to albumin was calculated using the comparative threshold cycle (CT) method and hCMV DNA was expressed as % virus bound (‘attachment’ step) or internalized (‘internalization’ step) using DMSO-treated samples as 100%.

### Animal Procedures:

Male and female Balb/c strain mice (6-8 weeks of age) from Taconic Biosciences Inc (Hudson, N.Y.) were maintained on a 12:12-h light cycle under constant temperature (22 ±2° C.) and humidity (55 ±5%), with access to food and water ad libitum. One to two females were cohabited with a male of the same strain for at least 1 week to ensure fertilization. When advanced pregnancy was seen, each pregnant female was caged singularly and checked for delivery twice daily, at 08:30 and 18:30. Focus was on inoculation of the newborn mouse, similar to the strategy recently described for studying the actions of Zika virus in the developing mouse brain (van Den Pol, et al., J Neurosci 37:2161-2175).

Paradigms of mCMV Infection:

Newborns were inoculated intraperitoneally (i.p.) with 750 plaque-forming units (PFU) of mCMV-GFP in 50 μL of media on the day of birth (DOB) within 14 hours of delivery. The DOB was considered postnatal day (P)0. Control animals received 50 μL of media i.p. To avoid any litter-size effect, large litters were culled to a maximum of 8-9 pups (Tanaka, et al., 1998, Reprod Toxicol 12:613-617). Infected and control pups were randomly assigned to receive VCD or vehicle (DMSO) via subcutaneous (s.c.) injections, once a day, at a dose of 1.4 mg/mL in 20 μL of saline (˜30 μg), starting after virus inoculation and running from P1 to P21. Mice were monitored daily for signs of mCMV-induced disease and to determine survival; weaning occurred on P21 and mice of either sex were housed separately until testing was completed, then sacrificed.

In addition to the intraperitoneal route, intracranial injection was performed in a group of newborn mice. Three days after birth, 2×10⁴ PFU of mCMV-GFP in 1 μL of media was injected into the left cerebral hemisphere of neonatal mice under cryoanesthesia using a 10-μl Hamilton syringe with a 32-gauge needle from a midpoint between the ear and eye. Infected pups were randomly assigned to receive daily doses of VCD or vehicle (DMSO), starting 3 hours after virus inoculation until P8. No deaths occurred and at P9 mice were euthanized and blood, liver, spleen, and brain were collected, snap-frozen, and stored at −80° C. until viral titer analysis via qPCR (n=8/experimental group) was performed.

Early Neurobehavioral Assessment

Intraperitoneally-infected pups and controls were assessed for neurobehavioral development according to a slightly modified Fox battery (Calamandrei, et al., 1999, Neurotoxicol. Teratol. 21:29-40). Evaluation was performed without knowledge of the experimental group on every other day from P2 to P14, in the light phase of the circadian cycle between 09:00 and 15:00. Each subject was tested at approximately the same time of the day. Reflexes and responses were scored in the following order:
Righting reflex: time employed by the pup to turn upright with all four feet when placed on its back;
Cliff aversion: when placed on the edge of a cliff or table top with the forepaws and face over the edge the mouse will turn and crawl away from the edge;
Forelimb grasping reflex: when the fore foot is stroked with a blunt instrument the foot will flex to grasp the instrument;
Forelimb placing reflex: contact of the dorsum of the foot against the edge of an object will cause the foot to raise and place itself on the surface of the object when the animal is suspended and no other foot is in contact with a solid surface;
Negative geotaxis: time employed by the pup to turn approximately 180° to either side when placed head down on a wire mesh screen (4×4 mm) held at a 45° angle;
Level screen test: pup holds onto a wire-mesh (10×10 cm) and is propelled across the mesh horizontally by the tail;
Screen climbing test: pup climbs up a vertical screen (10×10 cm, 90° angle) using both fore- and hind-paws. Maximal response is scored when the subject reaches the top of the vertical screen;
Vibrissa placing reflex: when the mouse is suspended by the tail and lowered so that the vibrissae make contact with a solid object, the head is raised and the forelimbs are extended to grasp the object.

Latencies were measured in seconds using a stopwatch for righting reflex and negative geotaxis. The remaining behavioral variables were rated semi-quantitatively in the following way: 0=no response or occurrence of the event (R/O), 1=slight/uncertain R/O, 2=incomplete R/O, and 3=a complete adult-like R/O. All timed responses were limited to a maximum of 60 seconds; therefore, absence of a milestone was scored as zero/60 seconds (semi-quantitative rating/latencies) if the mouse did not exhibit the behavior within 60 seconds.

This battery of tests provides a detailed assessment of functional and neuro-behavioral development throughout the neonatal period since the behaviors measured are each expressed at different stages of development during the first weeks of life. Specific information about vestibular function, motor development and activity, coordination, and muscle strength can be obtained by execution of these tests (St Omer, et al., 1991, Neurotoxicol. Teratol. 13:13-20; Schneider & Przewlocki, 2005, Neuropsychopharmacology 30:80-89).

Evaluation of Motor Coordination and Balance in Adolescent Mice:

Motor performance of infected and control mice, with or without VCD treatment, was assessed at P28-P30 by the hindlimb clasping, vertical pole, and challenging beam traversal test.

In the hindlimb clasping test, the mouse is gently lifted by the tail, grasped near its base, and the hindlimb position is observed for 10 seconds and scored as follows: if the hindlimbs are consistently splayed outward, away from the abdomen, it is assigned a score of 0; if one hindlimb is retracted toward the abdomen for more than 50% of the time suspended, it receives a score of 1; if both hindlimbs are partially retracted toward the abdomen for more than 50% of the time suspended, it receives a score of 2; if its hindlimbs are entirely retracted and touching the abdomen for more than 50% of the time suspended, it receives a score of 3 (Tanaka, et al., 2004, Nat. Med. 10:148-154).

The vertical pole test was conducted according to previously established protocols (Ogawa, et al., 1985, Res. Commun. Chem. Pathol. Pharmacol. 50:435-441). Briefly, mice were individually placed head-downward at the top of a vertical rough-surfaced pole (diameter, 8 mm; height, 55 cm) and allowed to descend in a round of habituation. Then, mice were placed head-upward at the top of the pole. The time required for the animal to descend to the floor was recorded as the locomotor activity time (T_{LA}), with a maximum duration of 120 seconds. If a mouse fell, was unable to turn downward or was unable to climb down, a default locomotor activity time value was recorded as 120 seconds. Each mouse was given three trials with a 30 second-recovery period between trials.

The challenging beam traversal test was performed as previously described (Fleming, et al., 2004, J. Neurosci. 24:9434-9440). The beam consisted of four sections (25 cm each, 1 m total length), each section having a different width. The beam started at a width of 3.5 cm and gradually narrowed to 0.5 cm in the last section. Underhanging ledges (1 cm width) were placed 1.0 cm below the upper surface of the beam to increase the sensitivity of the test and allow detection of subtle motor deficits (Brooks & Dunnett, 2009, Nat. Rev. Neurosci 10:519-529). Animals were trained to traverse the length of the beam starting at the widest section and ending at the narrow most difficult section. The narrow end of the beam led directly into the animal's home cage. A bright light illuminated the start of the beam to further encourage the mouse to walk across the beam towards the home cage. Animals received 2 days of training before testing, with 5 trials for each day. On the day of the test a mesh grid (1 cm squares) of corresponding width was placed over the beam surface leaving a 1 cm space between the grid and the beam surface. Animals were then videotaped while traversing the grid-surfaced beam for a total of five trials. Videotapes were viewed and rated in slow motion for hindlimb slips and time to traverse across five trials by an investigator blind to the mouse experimental group. A slip was counted when the mouse was facing and moving forward and a hindlimb slipped through or outside of the grid beyond 0.5 cm below the grid surface (halfway down).

Social Behavior and Exploratory Activity Analysis:

The exploratory activity was assessed in adolescent mice at P30-40 in an adapted small open field, as previously described (Schneider & Przewlocki, 2005, Neuropsychopharmacology 30:80-89). The apparatus consisted of a plastic rectangular box measuring 20.5×17×13 cm³ (1×w×h) with regularly spaced holes in the short (2) and long (3) walls, and illuminated by ambient fluorescent ceiling lights. The animal was placed in the center of the apparatus and its movements video-recorded over a 3-min period. Exploratory behavior was scored for number of rearing and nose-poking (nose of an animal put inside the hole) episodes.

Sociability and preference for social novelty were investigated at 5 weeks of age in a three-compartment apparatus (Yang, et al., 2011, Curr. Protoc. Neurosci. Chapter 8:Unit 8.26). Initially, test and control animals were allowed to explore the apparatus freely for a 10-min period (habituation). For the social approach paradigm, an unfamiliar conspecific (same sex, similar age and weight) animal was placed into one of the side compartments and restrained by a small wire object (‘social cage’). The compartment on the other side contained an empty wire object (‘empty cage’). The test subject was then released into the center compartment and allowed to explore the three-compartment apparatus freely for 10 min. Behavior was videotaped and assessed for the time(s) that the test subject spent in the three compartments and in close proximity to the social and empty cages. For the social-novelty paradigm, another unfamiliar conspecific animal was placed in the previously empty wire object (‘novel cage’). The behavior of the test mouse was recorded for 10 min and assessed for the time spent exploring the known and novel conspecifics.

Assessment of mCMV Distribution in the Brain and Viral-Mediated Brain Abnormalities:

At specific time-points after infection, mice were euthanized by an overdose of anesthetic and transcardially perfused with sterile, cold PBS followed by 4% paraformaldehyde, and brains harvested and weighed. Brains were then immersed overnight in 4% paraformaldehyde, cryoprotected in 15% and then 30% sucrose for 24 h before inclusion in Tissue Freezing Medium (General Data). Some i.p.-infected mice became dehydrated and moribund, and showed no sign of recovery; these mice were euthanized before the pre-defined sacrifice time-points and were recorded as having a lethal response to the virus.

Fifteen μm-thick sections cut with a Leica cryostat were employed for GFP reporter expression assessment and immunofluorescence analysis in the brain. Sections were dried for 4 hours at room temperature (RT), rehydrated in 1× PBS and then used for immunofluorescence assays. Briefly, tissue sections were incubated overnight at 4° C. with monoclonal mouse anti-NeuN antibody (1:500, EMD Millipore) for neuronal cells and polyclonal rabbit anti-calbindin D-28K (1:500, EMD Millipore) for cerebellar Purkinje cells. Tissues were washed three times in phosphate buffer plus 0.4% Triton-X. Secondary antibodies, including goat anti-mouse IgG and donkey anti-rabbit IgG conjugated to Alexa-594 (1:250) (Invitrogen), were applied for 1 h at room temperature and then washed off. Some sections were labeled with DAPI. Vectashield Fluorescent mounting medium (Vector Laboratories) was then used for mounting.

Images were collected by using a fluorescence microscope (IX 71; Olympus Optical, Tokyo, Japan). Frozen sections were used for morphometric measurements and cell numbers were quantified after imaging using Image J software. The molecular layer (ML) and internal granular layer (IGL) were assessed using images of serial mid-sagittal cerebellar sections stained with Calbindin D-28K and DAPI. Three measurements were taken at each side of the primary fissure in each section and 4 sections per animal were evaluated. For the cerebellar area, mid-sagittal brain sections (3 sections/mouse) were stained with blue fluorescent Nissl stain (NeuroTrace, ThermoFisher Scientific) and images were collected using a 2× objective. Cell count was performed on sections (4 sections/mouse) stained with Calbindin D-28K and the number of Purkinje cells was evaluated along 500 μm of the primary fissure (both sides). All measurements and quantifications were performed on at least 5 animals from 3 different litters.

Kinetics of Virus Spread and Replication in vivo:

For measurement of mCMV replication in blood, liver, spleen, and brain, mice infected i.p. or i.c. on the DOB and treated with VCD or vehicle were sacrificed at multiple time-points post-inoculation, and samples collected under sterile conditions, snap-frozen, and stored at −80° C. until viral titer analysis via quantitative real-time PCR (n=7-10/experimental group) was performed. Mice used for viral load analysis in liver, spleen, and brain were perfused with sterile cold PBS to remove any virus contained within the blood. Total DNA was isolated using the QIAamp DNA mini kit (Qiagen) as per the manufacturer's instructions. Quantitative PCR was performed using TaqMan assays (Life Technologies) by amplification of a fragment of mCMV IE1 gene exon-4 using the following primers; Forward: 5′-GGC TTC ATG ATC CAC CCT GTT A-3′ (SEQ ID NO:2) and Reverse: 5′-GCC TTC ATC TGC TGC CAT ACT-3′ (SEQ ID NO:3). The probe (5′-AGC CTT TCC TGG ATG CCA GGT CTC A-3′; SEQ ID NO:4) was labeled with the reporter dye FAM (Kosmac, et al., 2013, PLoS Pathog 9:e1003200). qPCR was carried out using 20-μL reaction mixtures employing the iTaq Universal SYBR Probes Supermix (BioRad) and 100 ng of DNA. Samples were run in duplicate using a two-step amplification protocol. Tissue samples from uninfected mice and samples without template served as negative controls. Viral burden was expressed as copy number per mL/gr of blood/tissue after comparison with a standard curve generated using serial ten-fold dilutions of mCMV DNA.

### Experimental Design and Statistical Analysis:

Statistical significance, unless otherwise specified, was determined by one-way Analysis of Variance (ANOVA) or Kruskal-Wallis test followed by Bonferroni's and Dunn's post-hoc test, respectively, for evaluation of motor performance, exploratory behavior, and brain morphometry. Early neurobehavioral ontogeny, social behavior, and viral load over time were assessed by a mixed-model ANOVA with Repeated Measures followed by Newman Keuls test if significant F-value. Since no gender-related differences were detected in early neurontogeny, data from male and female mice were combined. Only male mice were used for examination of motor performance and exploratory and social behavior. All analyses were conducted with GraphPad Prism 6.0, with significance set at p<0.05. Neurobehavioral assessment was performed blindly with respect to the experimental group.

### B. Discussion

Peripheral Inoculation of mCMV Causes Widespread Infection of the Developing Brain.

The kinetics of mCMV replication and dissemination after intraperitoneal inoculation (i.p.) of the virus in newborn mice on the day of birth (DOB, postnatal day 0) was characterized. Forty-eight hours after i.p. injection, mCMV was found in the blood and, at lower levels, in the spleen and liver of infected mice, with only a small amount detected in the brain (FIG. 12A). Analysis of viral kinetics in these four organs over the course of 50 days revealed that mCMV, after entering the bloodstream, quickly gained access to peripheral target organs, i.e. the liver and spleen, and began replicating to yield high viral titers by 4 days post-injection (dpi) (FIGS. 12B-12D). In turn, similar viral titers were measured in the brain only after 8 dpi (FIG. 12E). After entering the brain, the virus could effectively replicate in situ, as suggested by the measurement of mCMV loads similar to those found in the liver and spleen at the viral peak between P8 and P12 (FIGS. 12C-12E).

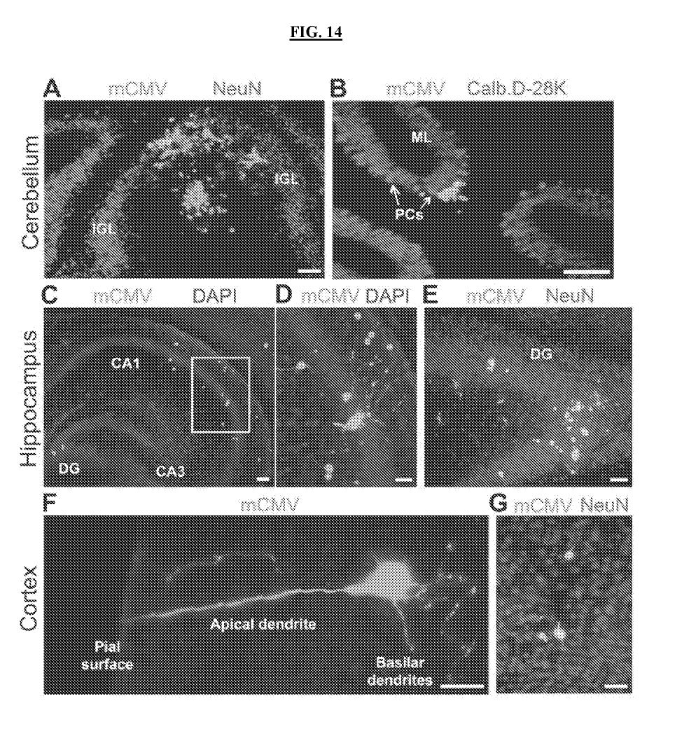

Upon histological examination, mCMV-GFP infection of the developing mouse brain appeared widespread and scattered in nature. Isolated infected cells and infectious foci containing up to 20-25 cells could be found in multiple distant areas within the same brain. The pattern of infection also appeared heterogeneous, with different brains displaying infection in different regions including the olfactory bulb and nuclei, the cortex, corpus callosum, hippocampus, basal nuclei, choroid plexus, midbrain, pons, cerebellum, and meninges (FIG. 13, panels A-I). No mCMV was detected in the spinal cord. Infection of the choroid plexus in the lateral ventricles was frequently associated with evidence of infected cells in the brain parenchyma in close proximity to the ventricle (FIG. 13, panels E-F), a site of neural progenitor stem cell localization. Infection of certain brain areas, such as the thalamus and the hypothalamus, was observed less frequently compared to other regions, including the cerebellum, hippocampus, and cortex. The cerebellum was the only site consistently displaying viral infection in all the brains examined (n=20), with robust GFP labeling in both Purkinje cells and granule neurons (FIG. 14, panels A-B). Viral GFP was also identified in neurons of the hippocampus and in the cerebral cortex (FIG. 14, panels C-G). In cortical pyramidal cells, GFP was seen in both the apical dendrite extending toward the cortical surface and in basal dendrites ramifying closer to the cell body. Some infected neurons in the cortex displayed signs of degeneration, characterized by abnormal swelling along the dendrites (FIG. 14, panel F).

Together, these results indicate that i.p. administered mCMV, after replicating in peripheral target organs, enters the developing brain of neonatal mice producing a scattered and widespread infection with a highly heterogeneous pattern of propagation. Nonetheless, mCMV appears to display a particular preference for the cerebellum as an infectious site.

Subcutaneous Valnoctamide Blocks mCMV Replication within the Brain

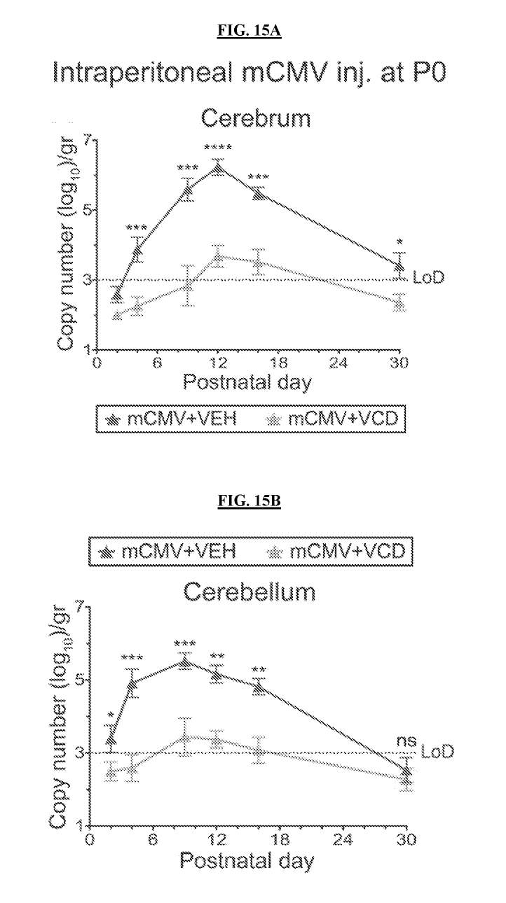

Mice were infected i.p. on the day of birth, and the brains of infected mice treated subcutaneously with VCD were compared with non-treated mice. CMV load in the brain was quantified at multiple time-points after virus inoculation. Cerebrum (cortex, hippocampus, thalamus, hypothalamus, striatum) and cerebellum were assessed separately to determine if the viral preference for the cerebellar region, as observed in the brain section analysis, was also accompanied by higher levels of virus replication. VCD decreased the amount of virus detected in both the cerebrum and cerebellum by a substantial amount, with an approximately 100- to 1000-fold decrease at all time-points tested (FIGS. 15A-15B). The anti-CMV effect displayed a rapid onset, suppressing the viral load after only 1 day and 3 days of treatment in the cerebellum and the cerebrum, respectively. In untreated infected mice, higher viral titers were identified in cerebellar samples compared to cerebrum at the beginning of infection (P4: t=3.704, p=0.004, paired Student's t-test), suggesting that the cerebellum may represent a preferential site for initial mCMV targeting in the brain. These data indicate that VCD can attenuate mCMV infection detected in the brain. The observed antiviral effect of VCD in the CNS could be the consequence of a drug-mediated decrease in viral replication in the periphery, i.e. liver and spleen, and therefore in the amount of virus that ultimately has access to the brain.

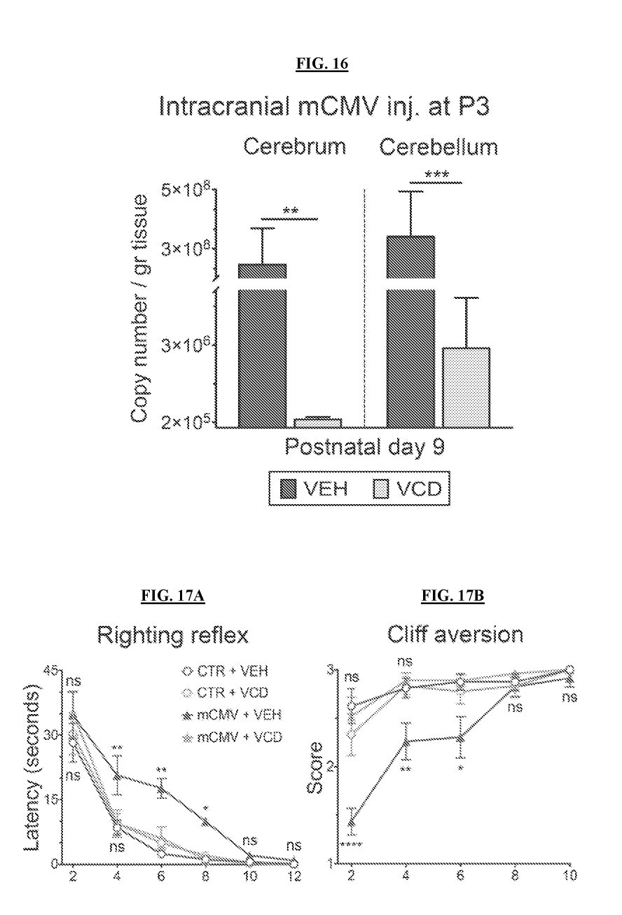

To investigate whether VCD can enter the CNS and act directly within the brain to block mCMV, pups on P3 were infected by direct intracranial inoculation. Analysis of mCMV load in the blood, liver, and spleen of untreated infected mice at P9 showed absence of viral spread outside the CNS. Viral titers in the cerebrum and the cerebellum were substantially lower by more than 100-fold in infected animals receiving VCD treatment compared to untreated controls (2.99×10⁵±9.06×10⁴ vs 2.47×10⁸±1.22×10⁸ copy number/g of tissue, p=0.004 in cerebrum; 2.88×10⁶±1.83×10⁶ vs 3.41×10⁸±1.52×10⁸ copy number/g of tissue, p=0.0003 in cerebellum; Mann-Whitney U-test) (FIG. 16). These results indicate that subcutaneously administered low-dose VCD can enter the brain at sufficient concentrations to effectively suppress mCMV replication in situ.

Early Neurological Dysfunction in mCMV-Infected Neonatal Mice Reversal

Human infants with CMV infection during early development can display substantial delays in the acquisition of neurological milestones during the first months of life. Since VCD showed a robust antiviral activity in the CNS of infected mice with a rapid attenuation of viral replication, it was investigated whether this would translate into a positive therapeutic effect on the early neurological outcomes of neonatal mice.

Neurobehavioral assessments were performed using a battery of tests to examine body righting and tactile reflexes, motor coordination, and muscular strength. These tests provide a detailed examination of neurontogeny throughout the neonatal period since the behaviors measured are each expressed at different periods during the first 3 weeks of postnatal life (Scattoni, et al., 2008, PLos One 3:e3067).

Here and in a number of experiments elsewhere herein, neurological function in 4 groups of mice were compared, including: non-infected controls, VCD-treated non-infected controls, CMV infected mice, CMV infected mice treated with VCD. VCD was administered in a single daily subcutaneous dose.

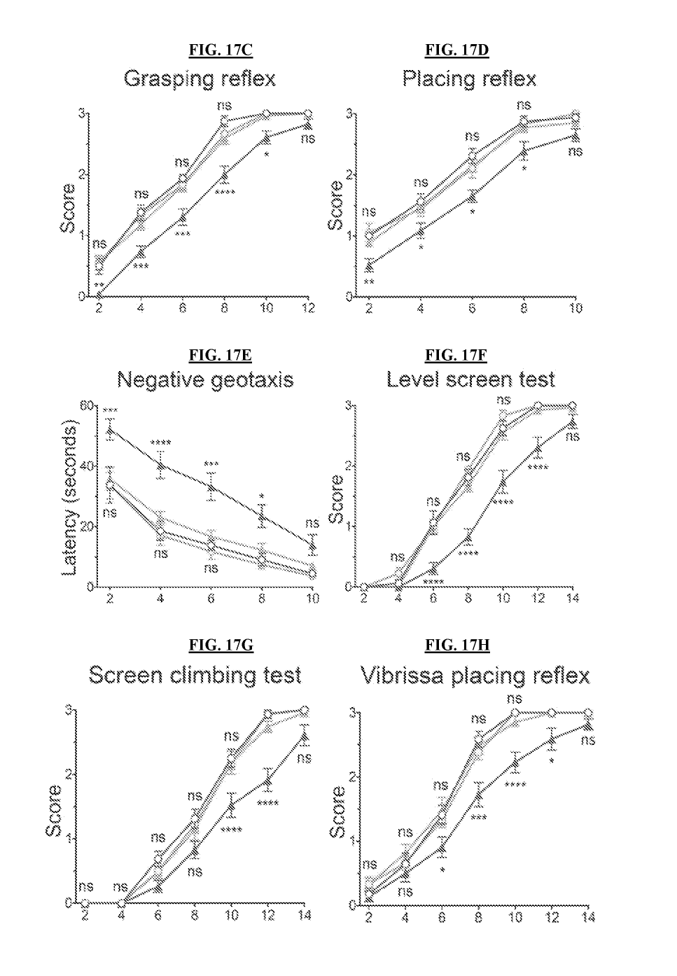

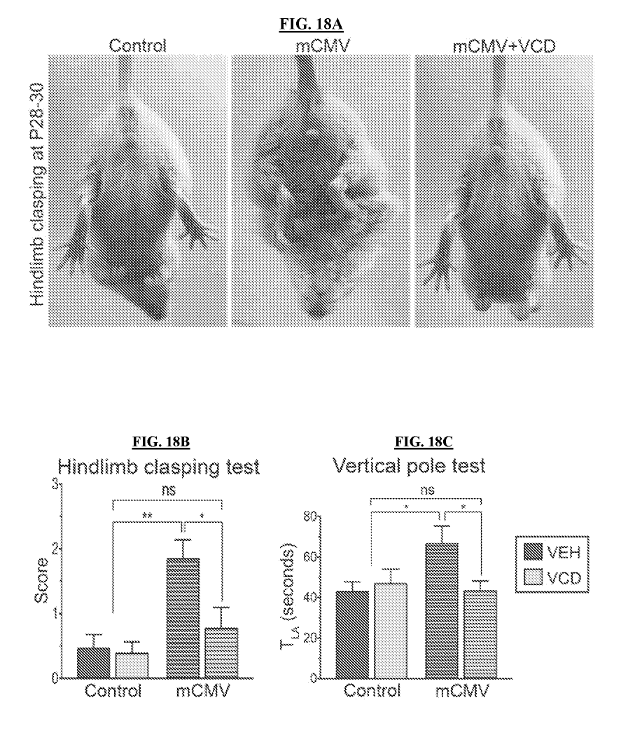

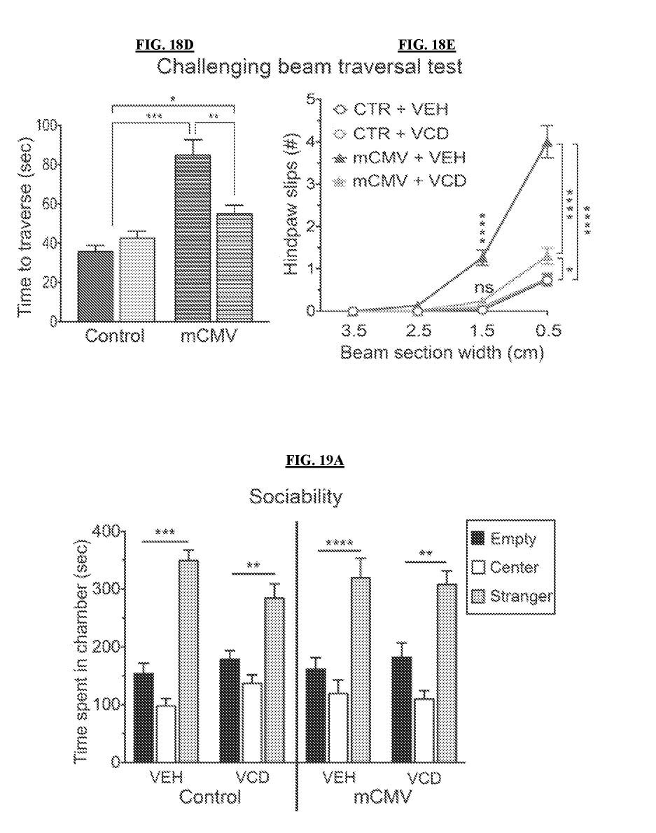

CMV infection on the day of birth induced abnormal acquisition of all the neurological milestones assessed, with infected mice showing a delay of 6 to 10 days in the demonstration of responses similar to the uninfected controls (FIGS. 17A-17H). In turn, infected VCD-treated neonatal pups displayed a timely acquisition of neurological milestones in all the behaviors measured. No differences were identified in the early neurontogeny of uninfected mice receiving VCD or vehicle. Together these data indicate that VCD treatment during early development can safely improve the short-term neurodevelopmental outcomes observed in infected neonatal mice.

### Amelioration of Long-Term Neurobehavioral Outcomes in Infected Juvenile Mice

CMV infected infants with evidence of neurological delays during the neonatal period are at increased risk of developing long-term permanent neurological and behavioral sequelae, which manifest with a delayed onset after the first years of life. Abnormal motor function is a commonly observed long-term neurological complication. More recently, a link between ASD-like behavioral disturbances in children and adolescents and CMV infection during early development has been proposed. Given the substantial improvement induced by VCD in the early neurontogeny of mCMV-infected neonatal mice, it was examined whether these beneficial effects could also ameliorate late-onset neurobehavioral abnormalities, including motor performance and social and exploratory behavior.

Motor Performance

The cerebellum appears to be a preferential site for mCMV targeting in the mouse brain. Cerebellar-mediated motor functions were investigated in infected and control juvenile mice using a hindlimb clasping test, a vertical pole test, and a challenging beam traversal test (Brooks & Dunnett, 2009, Nat. Rev. Neurosci. 10:519-529).

The hindlimb clasping test is a marker of cerebellar pathology commonly used for severity scoring in mouse models of cerebellar degeneration. The majority of the mCMV-infected mice (9/13) displayed an abnormal response to the clasping test, with both hindlimbs partially or entirely retracted to the abdomen when the mice were suspended by their tail for 10 seconds (FIGS. 18A-18B). VCD administration completely reversed this altered behavior, restoring a response similar to the uninfected counterparts.

By placing a mouse head upward on a vertical wooden pole, the vertical pole test allows for the examination of the ability of the animal to turn through 180° and successfully climb down the pole (Brooks & Dunnett, 2009, Nat. Rev. Neurosci. 10:519-529). mCMV-infected, untreated juvenile mice required a longer period to complete the task compared to both uninfected controls and mCMV-infected, VCD-treated animals (FIG. 18C). Three out of 20 (15%) infected mice without treatment failed the test, e.g. inability to turn head downward or falling, in all the three trials given, whereas no VCD-treated mice or uninfected controls failed in performing the task (p=0.03, Chi Square test).

In addition, fine motor coordination and balance were evaluated by the challenging beam traversal test, which assesses a mouse's ability to maintain balance while traversing a narrow, one-meter long beam to reach a safe platform (Brooks & Dunnett, 2009, Nat. Rev. Neurosci. 10:519-529). CMV infection during early development increased the time needed by the mice to cross the beam, and also the frequency of slipping (FIGS. 18D-18E). VCD treatment significantly improved the coordination and balance of mCMV-infected mice, reducing both the beam traversal time and the number of slips recorded.

Social and Exploratory Behavior

ASD is characterized by pervasive impairments in social interactions coupled with restricted and repetitive behaviors and decreased exploratory activity (American Psychiatry Association Diagnostic and Statistical Manual of Mental Disorders 5^{th} ed. 2013). To investigate whether adolescent mice with perinatal mCMV infection would display social and exploratory behavioral disturbances, social interaction and novel environment exploration were assessed by means of the three-chamber test and an adapted small open field test.

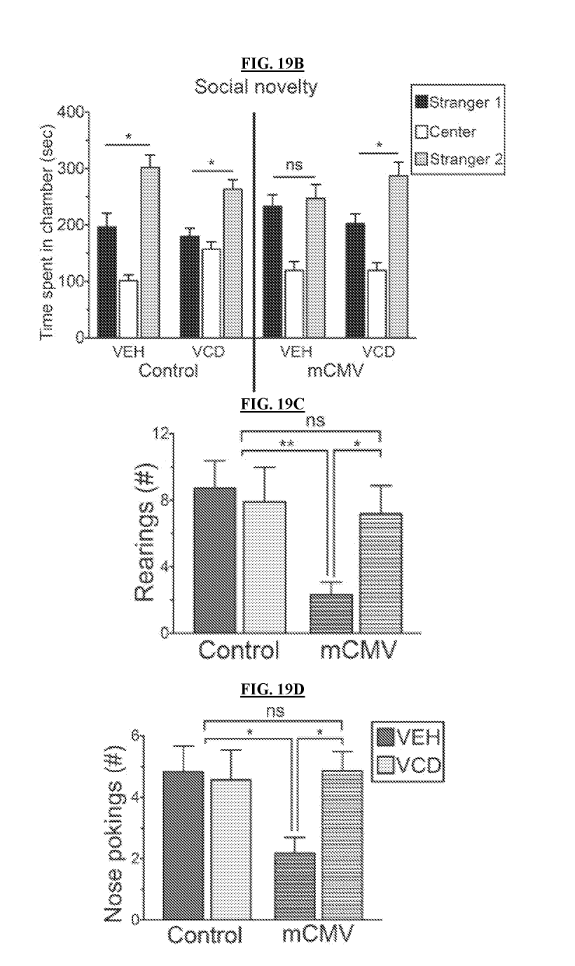

Infected untreated mice showed normal sociability when exposed to a first stranger mouse, preferring the conspecific over the empty cage (novel object) (FIG. 19A). However, lack of preference for social novelty was found when a second stranger was introduced, with infected untreated mice spending an equal amount of time in investigating the known and the novel animal (FIG. 19B). VCD therapy restored social novelty responses similar to levels shown in uninfected controls, with increased time devoted in examining the second stranger.

Exploratory activity was assessed by quantifying the number of rearings and nose-pokings of mice exposed to a novel environment over a 3 min-test session (FIG. 19C-19D). A substantial reduction in both rearing and hole-poking events was identified in mCMV-infected untreated mice as compared to control animals. Normal levels of exploratory activity were restored in infected mice receiving VCD treatment.

Valnoctamide Attenuates mCMV-Induced Brain Defects in Early Development

Early-onset neurodevelopmental delays and long-term permanent neurobehavioral disabilities are commonly observed in CMV-infected babies with evidence of virally induced brain abnormalities, including decreased brain size and cerebellar hypoplasia. Since VCD showed a potent and fast-acting anti-CMV activity in the brain of infected mice and appeared beneficial to both short- and long-term neurobehavioral outcomes, it was investigated whether drug-treatment during early development could also exert therapeutic actions on mCMV-induced brain defects.

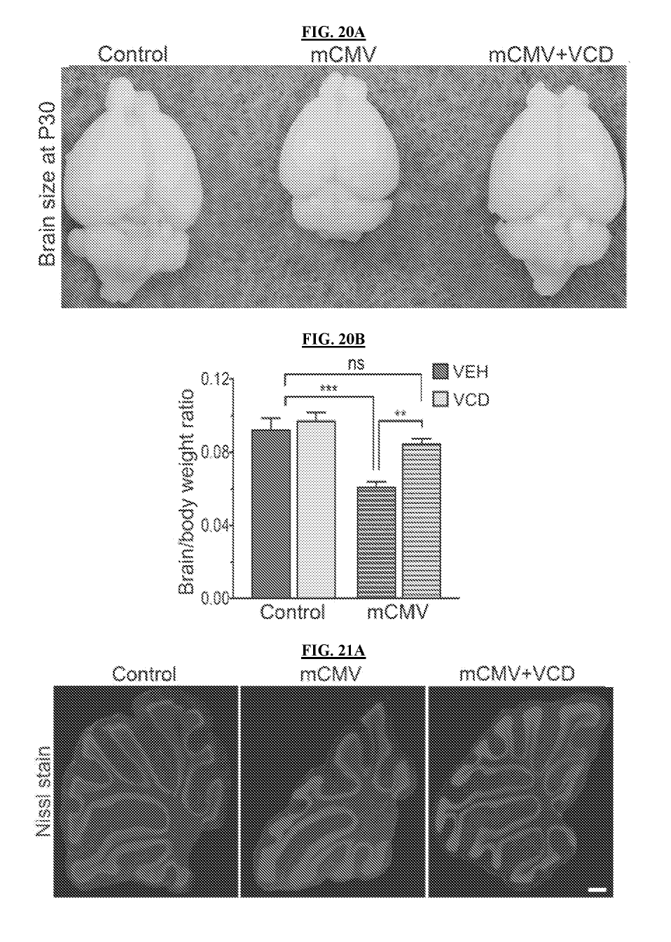

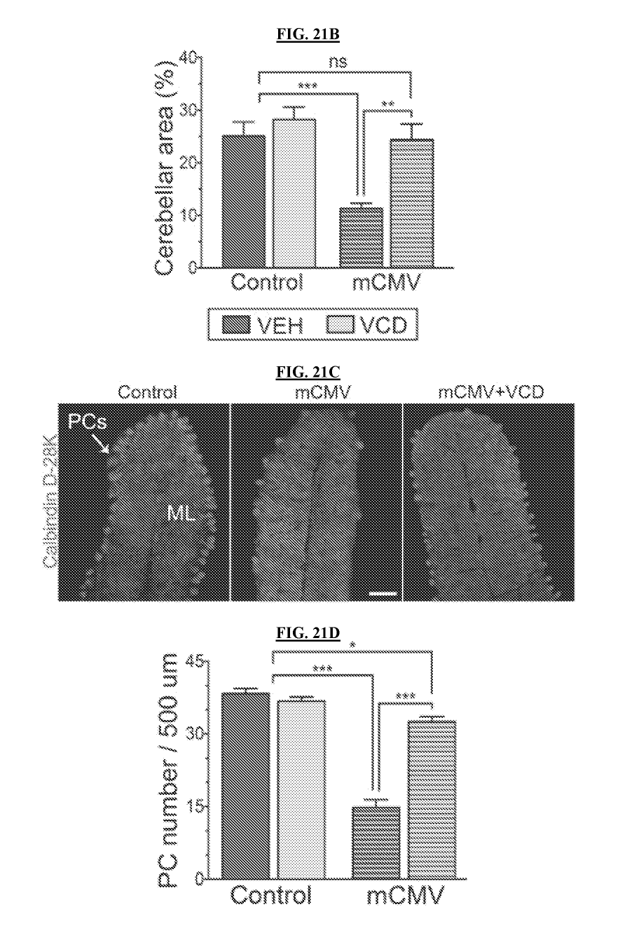

Brain size was analyzed in one month old-mice by assessing the brain to body weight ratio (FIGS. 20A-20B). This measurement allows a more objective evaluation of the postnatal brain growth, as compared to absolute brain weight, when somatic growth restriction is present. Subcutaneous VCD rescued the deficient brain growth induced by mCMV, restoring brain to body weight ratio values similar to uninfected control mice.

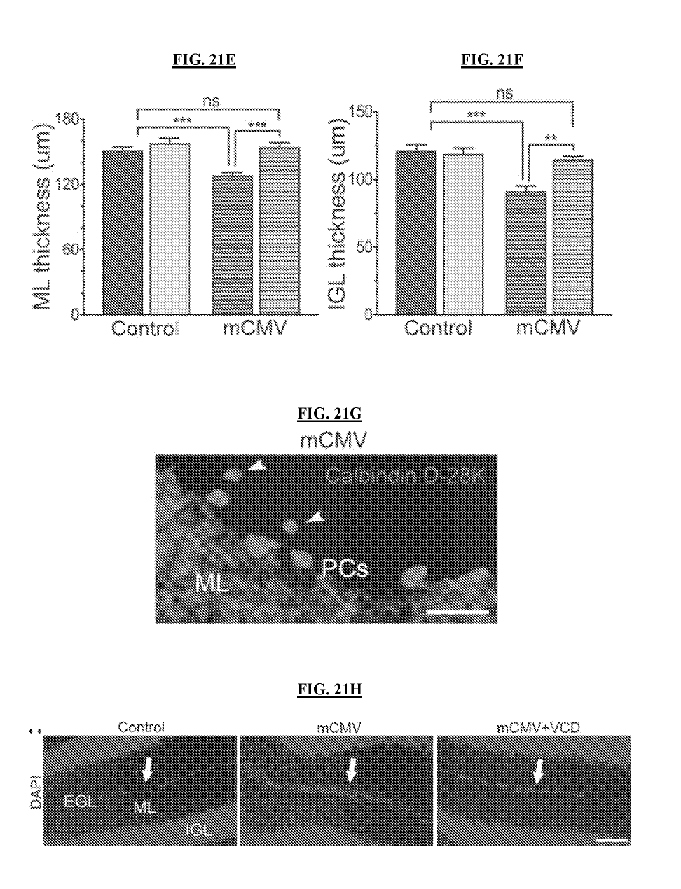

Hypoplasia of the cerebellum is a common radiological finding in CMV-infected human babies. A temporary delay in early postnatal cerebellar development was reported in newborn mice injected intraperitoneally with low titers of mCMV (Koontz, et al., 2008, J. Exp. Med. 205:423-435). In the present infected mice, the cerebellum was identified as a preferential site for viral localization in the brain. Cerebellar anatomy and histology were examined in control and infected mice with or without VCD therapy. mCMV infection of the developing brain resulted in the disruption of cerebellar development, with a 60% decrease in the total area of this region compared to uninfected controls (F=8.56, p<0.001 ANOVA) (FIGS. 21A-21B). mCMV infected mice displayed a substantial loss of Purkinje cells (PCs) and a thinner molecular layer (ML), which contains PC dendritic trees, parallel fibers of the granule cells, Bergmann glia radial processes, and basket and stellate cells (FIGS. 21C-21E). Reduced thickness of the cerebellar internal granular layer (IGL) was also found (FIG. 21F). PCs were not only decreased in number but also misplaced (FIG. 21G). In addition, the external granular layer (EGL), normally undetectable after P21 in rodent brains, could still be identified in mCMV-infected untreated mice at P30, whereas no EGL was visible in controls (FIG. 21H). Alignment of PCs and maturation of their dendritic trees, as well as granule cell precursor proliferation and inward migration from the EGL to the IGL, occur during the first 3 postnatal weeks of life in rodents. VCD treatment rescued the altered cerebellar development of infected animals, restoring normal cortical layer thickness and representation and markedly increasing PC number (FIGS. 21C-21H). These drug-mediated positive effects ultimately resulted in normalization of cerebellar size (FIGS. 21A-21B). No adverse side effects on either brain growth or morphometric parameters were detected in uninfected controls receiving VCD as compared to their vehicle-treated counterparts.

### Block of CMV Infection in Human Fetal Brain Cells

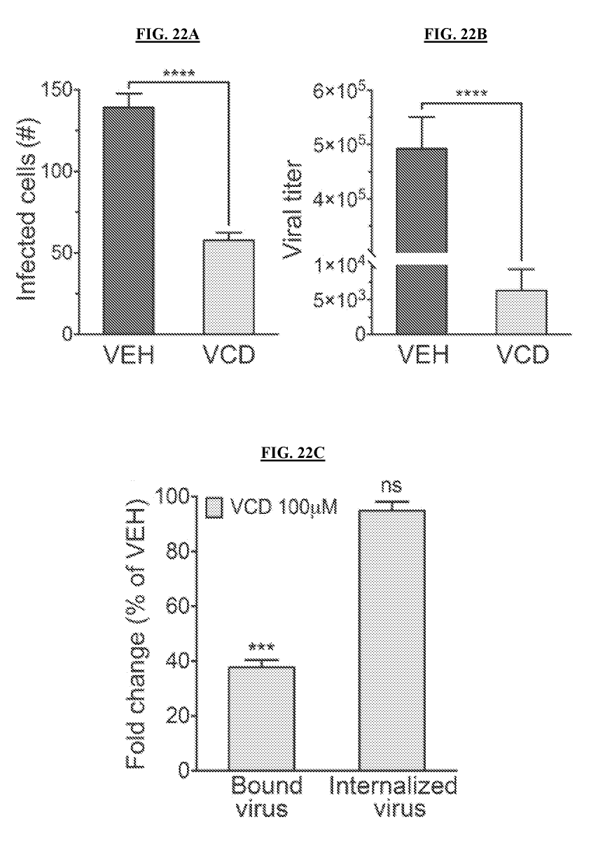

Mouse and human CMV share a close similarity in their viral genomes, but each retains species specificity. To corroborate that the results observed in the in vivo model with mCMV generalize to human CMV (hCMV), the actions of VCD were examined on human fetal astrocytes, a common cellular target which can play an important role in virus dispersal in the brain. VCD substantially decreased hCMV infectivity of fetal human astrocytes as assessed by quantification of cells expressing the CMV-GFP-reporter (FIG. 22A). Viral replication was also diminished in the presence of the drug, with a reduction in viral titer by approximately 100-fold (4.92×10⁵±5.84×10⁴ PFU/ml in vehicle-treated cultures vs 6.31×10³±3.06×10³ PFU/ml in VCD-treated cultures, p<0.0001, Mann-Whitney U-test) (FIG. 22B).

VCD appears to act at an early stage of CMV infection in fibroblasts, and has no antiviral effect on the unrelated vesicular stomatitis virus (Ornaghi, et al., 2016, Virology 499:121-135). To determine which step of the hCMV replication cycle was inhibited by VCD in human fetal astrocytes, a series of experiments were employed to assess virus attachment to the cellular surface and penetration into the cytoplasmic space. This was accomplished by shifting the incubation temperature from 4° C. (which allows virus attachment but not fusion and internalization) to 37° C. (which allows virus fusion and internalization). Viral genome quantification by qPCR showed that VCD appeared to block hCMV attachment to fetal astrocytes (FIG. 22C). In the presence of VCD, the amount of virus bound to the cell surface was decreased by 60% compared to control cultures not treated with VCD (p=0.0007, unpaired Student's t-test). VCD did not appear to block CMV fusion/internalization in the astrocytes. This also corroborates that the mechanism of VCD block of CMV occurs at an early stage of infection, and appears unrelated to the genomic mechanisms of other approved anti-CMV compounds.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating or preventing an infection by a Herpesviridae family virus in a mammalian subject, the method comprising administering to the subject a therapeutically effective amount of at least one compound selected from the group consisting of:
valnoctamide (2-ethyl-3-methylpentanamide) 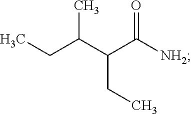
tert-butylethylacetamide (TED; 2-ethyl-3,3-dimethylbutanamide) 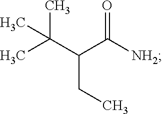
and
a molecule of formula (I): 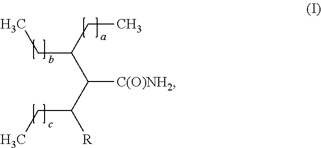
wherein
a is 0,1,2,3 or4;
b is 1, 2 or 3;
c is 1, 2 or 3; and
R is selected from the group consisting of H and C ₁-C₄ alkyl;
or a solvate, salt, enantiomer, or diastereoisomer thereof, or any mixtures thereof.

2. The method of claim 1, wherein a is 0.

3. The method of claim 1, wherein R is H or CH₃.

4. The method of claim 1, wherein one applies:
(a) R is H, a is 0, b is 1 and c is 1, and the compound is the molecule of structure (II), or a solvate, salt, enantiomer, or diastereoisomer thereof, or any mixtures thereof: 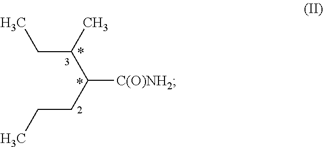
and
(b) R is CH₃, a is 0, b is 0 and c is 1, and the compound is the molecule of structure (III), or a solvate, salt, enantiomer, or diastereoisomer thereof, or any mixtures thereof:

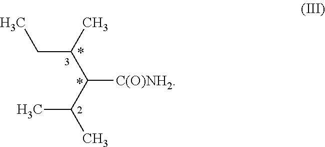

5. (canceled)

6. The method of claim 4, wherein (II) or (III) is a diastereoisomer selected from the group consisting of (2R,3R), (2R,3S), (2S,3R) and (2S,3S), or any mixtures thereof.

7. (cancelled)

8. The method of claim 1 any of claims 1, wherein the compound is in an enantiomerically or diastereoisomerically pure form.

9. The method of claim 1, wherein the compound is in an enantiomeric or diastereoisomeric mixture.

10. The method of claim 1, wherein the compound is part of a pharmaceutical composition or formulation further comprising at least a pharmaceutically acceptable carrier.

11. The method of claim 1, wherein the virus comprises at least one selected from the group consisting of cytomegalovirus (CMV), Epstein-Barr virus (EBV), herpes simplex virus (HSV) 1 and 2, varicella-zoster virus (VZV), and human herpes virus (HHV) 6, 7, and 8.

12. (canceled)

13. The method of claim 1, wherein the administration of the compound does not cause at least one of the following effects in the subject: (i) significant anticonvulsant effect and (ii) significant mood stabilizing or modifying effect.

14. (canceled)

15. The method of claim 1, wherein the subject is further administered one or more additional agents useful for treating or preventing the viral infection.

16. The method of claim 15, wherein the one or more additional agents comprise at least one selected from the group consisting of Ganciclovir, Valganciclovir, Foscarnet, Cidofovir, Fomivirsen, Aciclovir, and Valaciclovir.

17. (canceled)

18. (canceled)

19. The method of claim 1, wherein the compound is administered to the subject by at least one route selected from the group consisting of oral, nasal, inhalational, topical, buccal, rectal, pleural, peritoneal, intra-peritoneal, vaginal, intramuscular, subcutaneous, transdermal, epidural, intratracheal, otic, intraocular, intrathecal, intra-amniotic, intra-umbilical cord, and intravenous routes.

20. The method of claim 1, wherein the subject is human.

21. The method of claim 1, wherein the subject is pregnant, neonatal, or an unborn fetus.

22. (canceled)

23. The method of claim 21, wherein the subject is immunocompromised or the female carrying the fetus is immunocompromised.

24. (canceled)

25. (canceled)

26. A pharmaceutical composition comprising at least one compound selected from the group consisting of:
valnoctamide (VCD; 2-ethyl-3-methylpentanamide);
tert-butylethylacetamide (TED; 2-ethyl-3,3-dimethylbutanamide);
and
a molecule of formula (I): 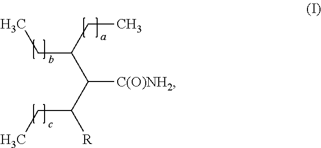
wherein
a is 0, 1, 2, 3 or 4;
b is 1, 2 or 3;
c is 1, 2 or 3; and
R is selected from the group consisting of H and C₁-C₄ alkyl,
or a solvate, salt, enantiomer, or diastereoisomer thereof, or any mixtures thereof;
and
one or more additional agents useful for treating or preventing a viral infection.

27. The pharmaceutical composition of claim 26, wherein the viral infection comprises a virus from the Herpesviridae family.

28. The pharmaceutical composition of claim 27, wherein the virus comprises CMV.

29. The pharmaceutical composition of claim 26, wherein that at least one compound is selected from the group consisting of valnoctamide, 2-ethyl-3,3-dimethylbutanamide (TED), 2-isopropyl-3-methylpentanamide (SID) and 3-methyl-2-propylpentanamide (SPD), or a solvate, salt, enantiomer, diastereoisomer or any mixtures thereof.


SEQUENCE LISTING (4 sequences)

SEQ ID NO: 1 - DNA, 21 bp, Artificial Sequence
  chemically synthesized
gcgtttgctcttcttcttgcg

SEQ ID NO: 2 - DNA, 22 bp, Artificial Sequence
  chemically synthesized
ggcttcatgatccaccctgtta

SEQ ID NO: 3 - DNA, 21 bp, Artificial Sequence
  chemically synthesized
gccttcatctgctgccatact

SEQ ID NO: 4 - DNA, 25 bp, Artificial Sequence
  chemically synthesized
agcctttcctggatgccaggtctca